(19) United States
(12) Patent Application Publication
(10) Pub. No.: US 2012/0323164 A1
(43) Pub. Date: Dec. 20, 2012

(54) PHOTODYNAMIC THERAPY WITH PHTHALOCYANINES AND RADICAL SOURCES

(75) Inventors: Malcolm E. Kenney, Cleveland Heights, OH (US); Yun Liu, Cleveland, OH (US)

(73) Assignee: Case Western Reserve University, Cleveland, OH (US)

(21) Appl. No.: 13/523,940

(22) Filed: Jun. 15, 2012

Related U.S. Application Data
(60) Provisional application No. 61/497,154, filed on Jun. 15, 2011.

(51) Int. Cl. A61M 37/00; A61P 35/00; A61P 17/06; A61P 17/00; A61K 31/695; C09B 47/04

(52) U.S. Cl. 604/20; 514/63; 540/128

(57) ABSTRACT

The use of phthalocyanines together with a free radical source for photodynamic therapy is described. The free radical sources cause the photodecomposition of the phthalocyanines, which can be useful for various reasons such as allowing light to penetrate to lower tissue levels that would otherwise be obscured. The nature of the phthalocyanine and the free radical source chosen can both have an influence on the rate of photodecomposition. The free radical sources can be provided along with the phthalocyanines either in free unattached form, or they can be attached to the phthalocyanines themselves.

### RELATED APPLICATIONS

This application claims priority to U.S. Provisional Application Ser. No. 61/497,154; filed on Jun. 15, 2011, which is incorporated herein by reference in its entirety.

### BACKGROUND OF THE INVENTION

Photodynamic therapy, hereinafter also referred to as “PDT”, is a process for treating various types of disease such as psoriasis and cancer wherein light irradiation is used to activate a substance, such as a dye or drug, which then attacks the target tissue through one or more photochemical reactions, thereby producing a cell-killing, or cytotoxic, effect. It has been discovered that when certain photosensitizer compounds are applied to the human or animal body, they are selectively retained by diseased (e.g., psoriatic or cancerous) tissue while being eliminated by healthy tissue. The diseased tissue containing the photosensitizer can then be exposed to therapeutic light of an appropriate wavelength and at a specific intensity for activation. The light energy, oxygen, and the photosensitizer cause a photochemical reaction which kills the cells in which the photosensitizer resides.

Phthalocyanines, hereinafter also abbreviated as “Pcs”, are a group of photosensitizer compounds having the phthalocyanine ring system. Phthalocyanines are azaporphyrins consisting of four benzoindole groups connected by nitrogen bridges in a 16-membered ring of alternating carbon and nitrogen atoms (i.e., C₃₂H₁₆N₈) which form stable chelates with metal and metalloid cations. In these compounds, the ring center is occupied by a metal ion (either a diamagnetic or a paramagnetic ion) that may, depending on the ion, carry one or two ligands. In addition, the ring periphery may be either unsubstituted or substituted. The synthesis and use of a wide variety of phthalocyanines in photodynamic therapy is described in International Publication WO 2005/099689. Phthalocyanines strongly absorb clinically useful red or near IR radiation with absorption peaks falling between about 600 and 810 nm, which potentially allows deep penetration of tissue by the light.

Phthalocyanines have also been used for additional purposes beyond PDT. For example, phthalocyanines have been used as pigments and dyes since 1936. In recent years, they have been widely studied for use in low-band-gap molecular solar cells, in optical switching and limiting devices, in thin-film semiconducting gas sensors, as photoconducting agents in photocopying machines, and as electrocatalysts.

Phthalocyanines are generally photostable. This photostability is typically advantageous in pigments and dyes and in many of the other applications of phthalocyanines However, it can be also be disadvantageous. For example, in PDT the phthalocyanine in the outer shell of a treated tumor can cause considerable opacity and prevent light from reaching the deeper tumor tissues. Patrice, T., Photodynamic Therapy. The Royal Society of Chemistry: London, 2003; Sobbi et al., J. Chem. Soc., Perkin Trans. 2, (3), 481-488 (1993). Therefore, controlled photodecomposition of PDT photosensitizers would increase the depth of penetration by irradiating light and thereby also increase treatment of deeper levels of tissue. On the other hand, if the photosensitizer decomposes too rapidly, cell destruction will not be complete. Thus, a slow photodecomposition rate of the photosensitizer is desirable in some PDT applications.

While phthalocyanines are photostable under many conditions, considerable photodecomposition of phthalocyanines during PDT has been reported. Lacey et al., Photochem. Photobio. Sci. 1, (2), 120-125 (2002); Bai et al., Photochem. Photobiol., 85, (4), 1011-1019 (2009). In one proposed mechanism of this photodecomposition, singlet oxygen (¹O₂) attacks the phthalocyanine and causes its decomposition. Maree et al., J. Photochem. Photobiol. A-Chem., 140, (2), 117-125 (2001). In another proposed mechanism radicals attack the phthalocyanine. Slota et al., Inorg. Chem., 42, (18), 5743-5750 (2003); Caronna et al., J. Photochem. Photobiol. A-Chem., 184, (1-2), 135-140 (2006). To further investigate these proposed mechanisms, the photostability of phthalocyanines in presence of ¹O₂ and free radical sources was studied.

### SUMMARY OF THE INVENTION

The phthalocyanines chosen for study were aluminum and silicon phthalocyanines, and the first radical source investigated was squalene. The results show that aluminum and silicon phthalocyanines decompose at a moderate rate in the presence of squalene, air and light. The rate of decomposition is influenced by the structure of the phthalocyanine. Generally phthalocyanines with smaller axial ligands decompose more rapidly. The decomposition can be inhibited by singlet oxygen quenchers and radical scavengers, and can be accelerated by oxygen. These results provide evidence for a mechanism which involves the generation of ¹O₂ from an excited phthalocyanine and ³O₂, the formation of free radicals by the reaction of the ¹O₂ with squalene, the attack of the radicals on the phthalocyanine ring, and finally the decomposition of the ring. The study also shows that the amino group in the aminosiloxysilicon phthalocyanine. Pc 4 plays an important role in its photodecomposition. The results suggest a mechanism which involves the formation of an exciplex by charge transfer between the ¹O₂ and the amino group, the generation of radicals from the decomposition of the exciplex, and the attack of the radicals on the phthalocyanine ring.

In further work, other alkenes were used as radical sources. The results show that the decomposition of phthalocyanines is influenced by the nature of the alkenes, including the activity of their allylic hydrogens, the number of double bonds in them, and their concentration.

With the knowledge obtained from the studies of phthalocyanine-alkene mixtures as a basis, a set of phthalocyanines bearing potential radical sources as ligands was synthesized and characterized by NMR, mass and UV-vis spectroscopy. Their photostability in air was investigated under white light. The silicon phthalocyanines studied include: carboxysilicon, alkoxysilicon, siloxysilicon, aminosiloxysilicon, methoxysilicon and thiomethoxysilicon phthalocyanines; and a set of Pc 12 salts. In addition an aminosiloxyaluminum phthalocyanine was studied.

The results show that the photostability of silicon and aluminum phthalocyanines can be controlled by linking radical sources to them. With double bonds as the radical sources, the decomposition rate is influenced by the number of double bonds, the distance between the double bonds and the ring, the presence or absence of conjugation in the double bonds, and the presence or absence of allylic hydrogen atoms.

With amino groups as radical sources, the photodecomposition rate is influenced by the number of amino groups, the distance between the amino groups and the ring, and the electron density on the amino nitrogen.

The possibility of using other hetero groups such as methoxy and thiomethoxy groups as radical sources was also explored. The results show that a methoxy group is less effective while a thiomethoxy is more effective in inducing the decomposition of silicon phthalocyanines. The effectiveness of these groups appears to be related to their basicity.

Pc 12 salts with unsaturated carboxylic acid anions were studied as yet another possible means of controlling the stability of phthalocyanines. The results show that their stability is similar to that of Pc 12.

An investigation on controlling the stability of aluminum phthalocyanines was started with a study of the aminosiloxyaluminum phthalocyanine. Pc 1. The results show that the photostability of Pc 1 is also influenced by its amino group, as is the case with Pc 4.

Accordingly, an improvement on PDT with phthalocyanines is the introduction of a free radical source such as an unsaturated adjuvant along with or sequential to phthalocyanine administration to adjust the light fluence, the phthalocyanine concentration, and the concentration of the unsaturated adjuvant so that ¹O₂-attack dominates and then radical-attack dominates, or so that they occur simultaneously. The free radical source can be introduced as a ligand replacing the OH group of the phthalocyanine, as a substituent replacing a hydrogen atom along the periphery of the phthalocyanine ring, or as an independent compound.

The procedure can provide destruction of cell organelles in the target area by two different routes: that by ¹O₂ and that by radicals. This can be advantageous as some types of cells are more susceptible to radicals than ¹O₂, or vice versa. An additional advantage is that providing a radical source can result in the destruction of the phthalocyanine and obviate the problems of phthalocyanine elimination from the body and the shielding of lower levels of target tissue from light by phthalocyanine.

### BRIEF DESCRIPTION OF THE DRAWINGS

The following is a brief description of the drawings which are presented for the purpose of illustrating the invention and not for the purpose of limiting it.

FIG. 1 provides a scheme showing a proposed mechanism of the decomposition of phthalocyanine by radical attack.

FIG. 2 shows the structures of various discrete alkene free radical sources.

FIG. 3 shows structures of saturated and unsaturated carboxysilicon phthalocyanines prepared.

FIG. 4 provides a scheme illustrating a proposed mechanism for the decomposition of unsaturated carboxysilicon phthalocyanines.

FIG. 5 shows the structures of alkoxysilicon phthalocyanines Pc 307, Pc 302, and Pc 303.

FIG. 6 shows structures of siloxysilicon phthalocyanines Pc 279, Pc 271, and Pc 270.

FIG. 7 shows the structures of several aminosiloxy silicon phthalocyanines studied.

FIG. 8 shows the structures of an aminosiloxysilicon, a methoxysiloxysilicon and a thiomethoxysiloxysilicon phthalocyanine.

FIG. 9 shows the structures of seven saturated and unsaturated carboxylic acid Pc 12 salts.

### DETAILED DESCRIPTION OF THE INVENTION

The present invention relates in part to the use of phthalocyanines together with a free radical source for photodynamic therapy. The free radical sources cause the photodecomposition of the phthalocyanines, which can be useful for various reasons such as allowing light to penetrate to lower tissue levels during photodynamic therapy. The nature of the phthalocyanine and the free radical source chosen can both have an influence on the rate of photodecomposition. The free radical sources can be provided along with the phthalocyanines either in free, unattached form, or they can be attached to the phthalocyanines.

### DEFINITIONS

The term “C_{x-y}acyl” refers to a group represented by the general formula:

C_{x-y}alkyl-C(O)—

The term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-trifluoroethyl, etc. C₀ alkyl indicates a hydrogen where the group is in a terminal position, a bond if internal. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively. When such alkenyl or alkynyl groups include more than one unsaturated bond, they can be referred to as polyunsaturated alkenyl or alkynyl groups.

The term “alkoxy” refers to an alkyl group having an oxygen attached thereto. Representative alkoxy groups include methoxy, ethoxy, propoxy, tert-butoxy and the like. An “ether” is two hydrocarbon groups covalently linked by an oxygen. Accordingly, the substituent of an alkyl that renders that alkyl an ether is or resembles an alkoxy.

The terms “amine” and “amino” are art-recognized and refer to both unsubstituted and substituted amines and salts thereof.

The term “aryl” as used herein includes 5-, 6-, and 7-membered substituted or unsubstituted single-ring aromatic groups in which each atom of the ring is carbon. Aryl groups include benzene, phenol, aniline, and the like.

The terms “carbocycle” and “carbocyclyl”, as used herein, refer to a non-aromatic substituted or unsubstituted ring in which each atom of the ring is carbon.

The terms “heteroaryl” includes substituted or unsubstituted aromatic 5- to 7-membered ring structures, more preferably 5- to 6-membered rings, whose ring structures include one to four heteroatoms. Heteroaryl groups include, for example, pyrrole, furan, thiophene, imidazole, oxazole, thiazole, triazole, pyrazole, pyridine, pyrazine, pyridazine and pyrimidine, and the like.

The term “heteroatom” as used herein means an atom of any element other than carbon or hydrogen. Preferred heteroatoms are nitrogen, oxygen, phosphorus, and sulfur.

The terms “heterocyclyl” or “heterocyclic group” refer to substituted or unsubstituted non-aromatic 3- to 10-membered ring structures, more preferably 3- to 7-membered rings, whose ring structures include one to four heteroatoms.

The terms identified above may be combined. For example, the term “C_{x-y}heteroaralkyl”, as used herein, refers to a C_{x-y}alkyl group substituted with a heteroaryl group.

The term “free radical source,” as used herein, refers to a compound that generates a substantial amount of free radicals upon exposure to singlet oxygen (¹O₂). Because double bonds provide a reaction site for ¹O₂, alkenyl compounds (including cycloalkenyl compounds) are suitable free radical sources, in particular alkenyl compounds including allylic alcohols and alkenes including a plurality of double bonds. Amine compounds can also serve as free radical sources. The free radical sources can be separate, discrete compounds, or they can be attached to another compound such as a phthalocyanine compound.

Another example of free radical sources includes polyunsaturated compounds such as polyunsaturated fatty acids. The term “polyunsaturated” refers to an alkenyl compound that contains more than one double bond. Fatty acids are carboxylic acids with an unbranched alkenyl group including at least four carbons. The multiple double bonds included in the alkenyl group can be either interrupted or conjugated. A polyunsaturated fatty acid attached by substitution may be referred to herein as a polyunsaturated C₄₋₂₄alkenyl ester.

The terms “PcIV” and “Pc 4”, as used herein represent a compound having a structure of Formula (I), wherein M is HOSiPcOSi(CH₃)₂(CH₂)₃N(CH₃)₂.

The term “substituted” refers to moieties having substituents replacing a hydrogen on one or more carbons of the framework. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

Substituents on fused ring structures can be peripheral or non-peripheral substituents. A non-peripheral substituent, as defined herein, is a substituent which is adjacent (i.e., α) to the point of fusion between an outer phenyl ring and an inner pyrrole ring, as found in phthalocyanine compounds as exemplified by Formula (I) herein. A substituent is peripheral, on the other hand, when it is positioned on an outer phenyl ring and is not adjacent to an inner pyrrole ring. For example, in Formula II provided herein, the substituents R², R³, R⁶, R⁷, R¹⁰, R¹¹, R¹⁴, and R¹⁵ are peripheral substituents.

As already noted herein, the photostability of photodynamic therapy photosensitizers such as Pc 4 can be disadvantageous if, for example, they shield the inner core of a tumor from light. Accordingly, an additional embodiment of the invention provides phthalocyanine photosensitizers with tunable photostability. By “tunable” photostability, what is meant is that the photostability can be varied (i.e., “tuned”) to a desired level by decreasing the photostability of a phthalocyanine photosensitizer by a desired amount. For example, the photostability can be decreased by an amount from about 10% to about 90%, (e.g., 20%, 30%, 40%, 50%, 60%, 70%, 80%, and intermediate amounts) relative to the photostability of an unmodified phthalocyanine. The photostability of the phthalocyanine can be varied in a number of ways, including, for example, changing the nature of the axial ligands, the nature of the central metal ion, or the strength of the associated free radical source.

One of the reasons for phthalocyanine's high photostability is that they are relatively resistant to photodegradation caused by singlet oxygen (¹O₂), but are less resistant to photodegradation caused by free radicals. Accordingly, the photostability of phthalocyanines can be adjusted by placing a source of free radicals that can be activated by light or other means in their vicinity. Upon activation, the free radical source will release free radicals which will then degrade the phthalocyanine compound. A variety of free radical sources can be used, as described herein. Phthalocyanine compounds with tunable photostability can include one or more free radical sources. For example, two free radical sources can be attached to a phthalocyanine compound by attaching a free radical source to each ligand bound to the central metal ion of the phthalocyanine compound, or multiple free radical sources can be substituted along the outside edge of the phthalocyanine ring. Alternately, the free radical source can be unattached to the phthalocyanine and provided as a discrete adjuvant.

A free radical source is placed in the vicinity of the phthalocyanine by attaching a free radical source to the phthalocyanine. The free radical source (e.g., a polyunsaturated compound) can be attached to the phthalocyanine in various different ways. For example, the polyunsaturated compound can be attached to the phthalocyanine through salt formation, ester formation, amide formation, or substitution. Salt formation typically occurs through association of an anion, such as that provided by a carboxyl group of a fatty acid, with a cationic amine group present in the phthalocyanine compound, such as one provided by an axial amine group. Ester formation, on the other hand, occurs at hydroxyl ligands such as those associated with the central metal in some phthalocyanine compounds or hydroxyl moieties found at the end of a ligand. Substitution can occur in various ways, and is exemplified by an amine, ether, carbon linking atom which attaches the free radical source to a peripheral or non-peripheral position on the phthalocyanine ring.

In one aspect, the present invention provides a composition comprising a phthalocyanine photosensiziter or a salt thereof and an unattached free radical source. The phthalocyanine and the unattached free radical source are provided in a single composition such that activation of the phthalocyanine by light of a suitable wavelength will also result in activation of the free radical source, leading to photodegradation of the phthalocyanine. Unattached, as used in this context, means that the free radical source is not chemically bonded to the phthalocyanine. Chemical bonding includes both covalent and ionic attachment.

In one embodiment of this aspect of the invention, the phthalocyanine compound has a structure of formula (I) or a salt thereof.

[Pc-M] (I)

wherein Pc is a substituted or unsubstituted phthalocyanine; and M is a diamagnetic metal ion, optionally complexed with or covalently bound to one or two axial ligands, wherein the metal ion is coordinated to the phthalocyanine moiety.

In a further embodiment, the phthalocyanine has a structure of formula (II) or a salt thereof

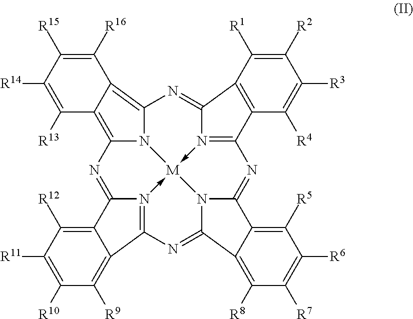

wherein M is a diamagnetic metal ion optionally complexed with or covalently bound to one or two axial ligands, wherein the metal ion is coordinated to the phthalocyanine moiety.

In formula II of this embodiment, R¹-R¹⁶ are each independently selected from hydrogen, halogen, nitro, cyano, hydroxy, thiol, amino, carboxy, aryl, heteroaryl, carbocyclyl, heterocyclyl, C₁₋₂₀alkyl, C₁₋₂₀alkenyl, C₁₋₂₀alkynyl, C₁₋₂₀alkoxy, C₁₋₂₀acyl, C₁₋₂₀alkylcarbonyloxy, C₁₋₂₀aralkyl, C₁₋₂₀hetaralkyl, C₁₋₂₀carbocyclylalkyl, C₁₋₂₀heterocyclylalkyl, C₁₋₂₀aminoalkyl, C₁₋₂₀alkylamino, C₁₋₂₀thioalkyl, C₁₋₂₀alkylthio, C₁₋₂₀hydroxyalkyl, C₁₋₂₀alkyloxycarbonyl, C₁₋₂₀alkylaminocarbonyl, C₁₋₂₀alkylcarbonylamino, C₁₋₁₀alkyl-Z—C₁₋₁₀alkyl; R¹⁷ is selected from hydrogen, C₁₋₂₀acyl, C₁₋₂₀alkyl, and C₁₋₂₀aralkyl; and Z is selected from S, NR¹⁷, and O.

In another embodiment, the compound of formula II, as defined above, is further defined such that the axial ligand M is (G)aY[(OSi(CH₃)₂(CH₂)_{b}N_{c}(R′)_{d}(R″)ₑ)_{f}Xg]p; Y is selected from Si, Al, Ga, Ge, or Sn; R′ is selected from H, CH₃, C₂H₅, C₄H₉, C₄H₈NH, C₄H₈N, C₄H₈NCH₃, C₄H₈S, C₄H₈O, C₄H₈Se, OC(O)CH₃, OC(O), CS, CO, CSe, OH, C₄H₈N(CH₂)₃CH₃, (CH₂)₂N(CH₃)₂, (CH₂)ₙN((CH₂)ₒ(CH₃))₂, and an alkyl group having from 1 to 12 carbon atoms; R″ is selected from H, SO₂CH₃, (CH₂)₂N(CH₃)₂, (CH₂)₁₁CH₃, C(S)NHC₆H₁₁O₅, (CH₂)ₙN((CH₂)ₒ(CH₃))₂, and an alkyl group having from 1 to 12 carbon atoms; G is selected from OH and CH₃;

In this embodiment, the compound of formula II is further defined such that X is selected from I, F, Cl, or Br; a is 0 or 1; b is an integer from 2 to 12; c is 0 or 1; d is an integer from 0 to 3; e is an integer from 0 to 2; f is 1 or 2; g is 0 or 1; n is an integer from 1 to 12; o is an integer from 1 to 11; and p is 1 or 2. In some embodiments, the compounds include only those in which Y is Si or Al.

In a further embodiment of the invention, the phthalocyanine compounds of formula II include only those in which R¹, R⁴, R⁵, R⁸, R⁹, R¹², R¹³, and R¹⁶ are each independently selected from hydrogen, halogen, nitro, cyano, hydroxy, thiol, amino, and methyl; and R², R³, R⁶, R⁷, R¹⁰, R¹¹, R¹⁴, and R¹⁵ are each independently selected from hydrogen, halogen, nitro, cyano, hydroxy, thiol, amino, carboxy, aryl, heteroaryl, carbocyclyl, heterocyclyl, C₁₋₆alkyl, C₁₋₆alkenyl, C₁₋₆alkynyl, C₁₋₆alkoxy, C₁₋₆acyl, C₁₋₆alkylcarbonyloxy, C₁₋₆carbocyclylalkyl, C₁₋₆aminoalkyl, C₁₋₆alkylamino, C₁₋₆thioalkyl, C₁₋₆alkylthio, C₁₋₆hydroxyalkyl, C₁₋₆alkyloxycarbonyl, C₁₋₆alkylaminocarbonyl, and C₁₋₆alkylcarbonylamino. In addition, in some cases, the compounds may include only those in which M is selected from HOSiOSi(CH₃)₂(CH₂)₃N(CH₃)₂, Si[OSi(CH₃)₂(CH₂)₃N(CH₃)₂]₂, Si[OSi(CH₃)₂(CH₂)₃OCH₃]₂, and Si[OSi(CH₃)₂(CH₂)₃SCH₃]₂.

In embodiments of the invention in which the free radical source is provided separate from the phthalocyanine, the free radical source can be a polyunsaturated alkene or cycloalkene. For example the free radical source can be selected from compounds including 1,4-cyclohexadiene, 1,3-cyclooctadiene, 1,5-cyclooctadiene, linoleic acid, geraniol, farnesol, and squalene.

In some embodiments of the invention, the phthalocyanine and the free radical source are provided in compositions that are not necessarily pharmaceutically acceptable. For example, the phthalocyanine and the free radical source may be used together to provide an autodegrading dye. However, in other embodiments of the invention, the composition used is a pharmaceutical composition including a phthalocyanine salt that is a pharmaceutically acceptable salt, and a pharmaceutically acceptable carrier.

For pharmaceutical compositions, the anion Y attached to the ligand M in formula II can selected from the group consisting of bromide, chloride, sulfate, bisulfate, phosphate, nitrate, acetate, pyruvate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate anions. Examples of pharmaceutical compositions including the phthalocyanine and a free radical source include systemic formulations and topical formulations.

Another aspect of the invention provides a method of photodynamic therapy using a phthalocyanine photosensitizer together with an unattached (i.e., discrete) free radical source. The method includes the steps of administering a phthalocyanine photosensitizer to the subject, administering a discrete free radical source to the subject, and irradiating a target tissue in the subject with light having a wavelength suitable for excitation of the phthalocyanine photosensitizer. The phthalocyanine photosensitizer and the free radical source can be coadministered, administered at the same time, or at different times. In some embodiments, the target tissue is psoriatic tissue, eczemous tissue, or a tumor. In other embodiments, the target tissue is infected tissue such as tissue infected by a fungal infection, a viral infection, or a bacterial infection. The phthalocyanine used can be selected from any of the embodiments of the phthalocyanine described herein. A variety of suitable free radical sources can be used as well. For example, the free radical source can be selected from 1,4-cyclohexadiene, 1,3-cyclooctadiene, 1,5-cyclooctadiene, linoleic acid, geraniol, farnesol, and squalene.

In another aspect of the invention, a phthalocyanine photosensitizer is provided in which the phthalocyanine compound has a structure of formula (I) or a salt thereof.

[Pc-M] (I)

wherein Pc is a substituted or unsubstituted phthalocyanine; and M is a diamagnetic metal ion, optionally complexed with or covalently bound to one or two axial ligands, wherein the metal ion is coordinated to the phthalocyanine moiety, and wherein the phthalocyanine compound further includes a free radical source that is chemically attached to the phthalocyanine compound.

In a further embodiment of the invention directed to phthalocyanines including one or more attached free radical sources, the phthalocyanine photosensitizer has a structure according to formula (II):

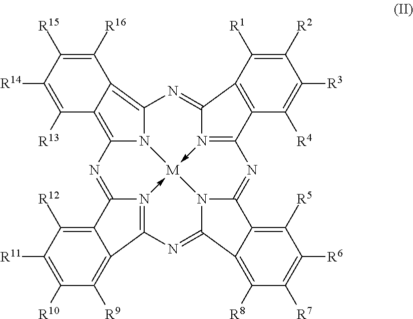

wherein M is a diamagnetic metal ion optionally complexed with or covalently bound to one or two axial ligands, wherein the metal ion is coordinated to the phthalocyanine moiety; and R¹-R¹⁶ are each independently selected from hydrogen, halogen, nitro, cyano, hydroxy, thiol, amino, carboxy, aryl, heteroaryl, carbocyclyl, heterocyclyl, C₁₋₂₀alkyl, C₁₋₂₀alkenyl, C₁₋₂₀alkynyl, C₁₋₂₀alkoxy, C₁₋₂₀acyl, C₁₋₂₀alkylcarbonyloxy, C₁₋₂₀aralkyl, C₁₋₂₀hetaralkyl, C₁₋₂₀carbocyclylalkyl, C₁₋₂₀heterocyclylalkyl, C₁₋₂₀-aminoalkyl, C₁₋₂₀alkylamino, C₁₋₂₀alkylthio, C₁₋₂₀hydroxyalkyl, C₁₋₂₀alkyloxycarbonyl, C₁₋₂₀alkylaminocarbonyl, C₁₋₂₀alkylcarbonylamino, C₁₋₁₀alkyl-Z—C₁₋₁₀alkyl; R¹⁷ is selected from hydrogen, C₁₋₂₀acyl, C₁₋₂₀alkyl, and C₁₋₂₀aralkyl; and Z is selected from S, NR¹⁷, and O, and wherein the free radical source is selected from unsaturated carboxy ligands, unsaturated alkoxy ligands, unsaturated siloxy ligands, and thiomethoxysiloxy ligands and is substituted on one or more of the axial ligands or R¹-R¹⁶.

Another aspect of the invention provides a method of photodynamic therapy using phthalocyanine compounds with one or more attached free radical sources. The method includes the steps of administering a phthalocyanine photosensitizer including a free radical source to the subject and irradiating a target tissue in the subject with light having a wavelength suitable for excitation of the phthalocyanine photosensitizer. In embodiments of this method, the target tissue can be psoriatic tissue, eczemous tissue, or a tumor. Target tissue can also be tissue that is infected, including fungal infection, viral infection, and bacterial infection. The phthalocyanines used in this method can be any of the phthalocyanine photosensitizers including attached free radical sources that are described herein.

A “therapeutically effective amount” of a compound with respect to the subject method of treatment, refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” or “treatment” includes reversing, reducing, or arresting the symptoms, clinical signs, and underlying pathology of a condition in manner to improve or stabilize a subject's condition.

Methods for conducting photodynamic therapy are known in the art. See for example Thierry Patrice. Photodynamic Therapy; Royal Society of Chemistry, 2004. The phthalocyanines and free radical sources can be applied separately or together to a target tissue as a step in photodynamic therapy. In certain embodiments, the composition is applied to an epithelial, mesothelial, synovial, fascial, or serosal surface, including, but not limited to, the eye, esophagus, mucous membrane, bladder, joint, tendon, ligament, bursa, gastrointestinal, genitourinary, pleural, pericardial, pulmonary, or uroepithelial surfaces. In certain embodiments, the composition is applied to the surface of the skin. Alternately, the phthalocyanines and the free radical sources can be delivered systemically, relying on preferential accumulation in diseased tissue and/or exposure to activating radiation to provide a selective effect on diseased tissue.

Photodynamic therapy can be used to treat a variety of different diseases and conditions. Examples of diseases treatable by photodynamic therapy include psoriasis, eczema, and various infections including fungal infections, viral infections, and bacterial infections. Photodynamic therapy can also be used for photorejuvincation or for aesthetic dermatological treatment In accordance with the present invention, these diseases or conditions can be treated using a combination of phthalocyanines with free radical sources which are either attached to the phthalocyanines or administered as separate compounds. Administration of phthalocyanine together with a free radical source can also be used to purge bone marrow for autologous bone marrow transplantation, purge viruses from whole blood or blood components, treat warts, treat macular degeneration, or treat intra-arterial plaques.

Another aspect of the invention relates to a method for treating cancer comprising administering a pharmaceutical composition including a phthalocyanine and a free radical source to cancerous tissue or a surface of cancerous tissue and irradiating the surface. Cancer, as used herein, refers to a disease of abnormal and excessive cell proliferation, as known by those skilled in the art, and also includes precancerous conditions. The surface can be skin in the case of skin cancer, or an exposed internal surface in the case of other types of cancer. Skin cancers include, but are not limited to basal cell carcinoma, squamous cell carcinoma, and melanoma.

The term “subject” for purposes of treatment includes any human or animal subject who has a disorder amenable to treatment by photodynamic therapy. Besides being useful for human treatment, the compounds of the present invention are also useful for veterinary treatment of mammals, including companion animals and farm animals, such as, but not limited to dogs, cats, horses, cows, sheep, and pigs. In most embodiments, the subject is human.

The present invention provides various phthalocyanine or phthalocyanine salt compositions that can be used to prepare formulations for systemic or topical administration. If discrete free radical sources are used, they can be formulated either together or separately from the phthalocyanine or phthalocyanine salt. Systemic administration includes delivery of an aqueous solution, preferably a buffered aqueous solution, including a phthalocyanine salt. Systemic formulations typically also include a dispersant. Systemic administration is typically done parenterally (e.g., intravenously or intramuscularly). However, systemic administration can also be carried out by oral administration.

Topical administration of phthalocyanines or phthalocyanine salts can be accomplished using various different formulations such as powders, sprays, ointments, pastes, creams, lotions, gels, solutions, or patches. The active component may be mixed under sterile conditions with a pharmaceutically acceptable carrier, and with any preservatives, buffers, or propellants which may be required. The ointments, pastes, creams, solutions, foams, lacquers, oils and gels may contain excipients in addition to phthalocyanine(s). These formulations may contain a phthalocyanine salt within or on micro or nanoparticles, liposomes, beads, polymer matrices, sponges, osmotic pumps, or other structures.

Phthalocyanines or phthalocyanine salts can be formulated as ointments or creams for topical administration. Ointments are homogeneous, semi-solid preparations intended for external application to the skin or mucous membranes. They are used as emollients or for the application of active ingredients to the skin for protective, therapeutic, or prophylactic purposes and where a degree of occlusion is desired. Ointments can be formulated using hydrophobic, hydrophilic, or water-emulsifying bases to provide preparations for various applications. Creams, on the other hand, are semi-solid emulsions; i.e., a mixture of oil and water. They are divided into two types: oil-in-water creams which are composed of small droplets of oil dispersed in a continuous aqueous phase, and water-in-oil creams which are composed of small droplets of water dispersed in a continuous oily phase.

Phthalocyanines and phthalocyanine salts can also be administered by aerosol. This is accomplished by preparing an aqueous aerosol, liposomal preparation, or solid particles containing the compound. A nonaqueous (e.g., fluorocarbon propellant) suspension could be used. Sonic nebulizers are preferred because they minimize exposing the agent to shear, which can result in degradation of the compound. Ordinarily, an aqueous aerosol is made by formulating an aqueous solution or suspension of the agent together with conventional pharmaceutically acceptable carriers and stabilizers.

As described herein, phthalocyanines and phthalocyanine salts can also be formulated for delivery as a gel. Gel formulations comprising a phthalocyanine salt may be prepared according to U.S. Pat. No. 6,617,356 or U.S. Pat. No. 5,914,334, the disclosures of which are incorporated herein in their entirety. In addition, phthalocyanine-containing gells can be dried to form films suitable for phthalocyanine administration.

Transdermal patches have the added advantage of providing controlled delivery of a phthalocyanine to the body. Such dosage forms can be made by dissolving or dispersing the agent in the proper medium. Absorption enhancers can also be used to increase the flux of the photosensitizer(s) into the skin. The rate of such flux can be controlled by either providing a rate controlling membrane or dispersing the agent in a polymer matrix or gel.

Phthalocyanine formulations can also be delivered transdermally using microneedles. See for example Arora et al., International Journal of Pharmaceutics, 364, pg. 227-236 (2008), which describes micro-scale devices for transdermal drug delivery.

Delivery of phthalocyanines across an epithelial, epidermal, serosal or mucosal surface may be accomplished using application of an electrical current and a charged solvent solution, such as iontophoresis.

“Pharmaceutically acceptable carrier” as used herein means a pharmaceutically acceptable material, composition or vehicle, such as a liquid or solid filler, diluent, excipient, solvent or encapsulating material, involved in carrying or transporting the subject chemical from one organ or portion of the body, to another organ or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose, and sucrose; starches, such as corn starch and potato starch; cellulose, and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

Several examples have been included to more clearly describe particular embodiments of the invention, such as the specific process utilized to synthesize the phthalocyanine salts. However, there are a wide variety of other embodiments within the scope of the present invention, which should not be limited to the particular example provided herein.

### EXAMPLES

### Example 1

### Photostability of Al and Si Phthalocyanines to Free Radicals

With the previous work showing that aluminum and silicon phthalocyanines are stable to ¹O₂, attention naturally turns to the resistance of these phthalocyanines to free radicals. As a source of free radicals, the reaction of polyunsaturated alkenes with ¹O₂ (from irradiation of the phthalocyanines) was chosen. Various polyalkenes were considered. Ultimately squalene was chosen because it reacts readily with singlet oxygen to give free radicals and is commercially available. In addition, it is easily soluble in toluene and has an uncomplicated structure (no functional groups other than double bonds).

For all of the photodecomposition experiments, a slide projector (EKTAGRAPHIC III E, Kodak, Japan) equipped with a tungsten-halogen lamp (300 W, EIKO, Shawnee, Kans.) powered to operate at 3350 K, and a custom-made polypropylene cuvette holder were used. The distance between the cuvette face and the lamp filament was 30 cm. Determination of the beam intensity at the cuvette face was made with a powermeter (Powermax, pm10, Coherent, Santa Clara, Calif.). The beam intensity was checked before each measurement.

Solutions for the photodecomposition experiments were prepared as follows. In a typical solution preparation, the phthalocyanine (2.0 mg) was dissolved with sonication (Branson 2510, Branson Ultrasonics, Danbury, Conn.) in toluene (4.0 mL) to give a solution containing 0.5 mg/mL. The extinction coefficients, ε, of the Q bands in toluene of all the silicon phthalocyanines studied were assumed to be 2.5×10⁵, the extinction coefficient of SiPc(OSiHx₃)₂, Pc 162, in toluene. This assumption was justified on the basis that all of the silicon phthalocyanines studied contained simple, unsubstituted phthalocyanine rings. The extinction coefficients of the aluminum phthalocyanines were assumed to be 2.3×10⁵, the extinction coefficient of AlPcOSi(CH₃)₂(CH₂)₃N(CH₃)₂, Pc 1.

The absorbance of the Q band of the solution, A, was determined with a spectrophotometer (Lambda 25, Perkin Elmer, Shelton, Conn.; fused silica cuvettes, 1.00 cm, 3.5 mL, Fisher Scientific). From A, ε, the cuvette path length, b, and Beer's law, the concentration of the solution, c, was determined. The solution was then diluted with toluene to a concentration of 9.2 μM. To verify that this was the concentration of the solution, its absorbance was measured again. If its absorbance was within ±0.05 of 2.30, that is, if it was within ±0.15 of the absorbance of a 9.2 μM solution of Pc 162, it was used.

Photodegradation Determinations.

In a typical determination, the solution (3.00 mL) was placed in a cuvette (1.00 cm, 3.5 mL, Fisher Scientific, Pittsburgh, Pa.) and its absorbance was measured with the spectrometer (Lambda 25) before irradiation and at 2, 4, 6 and 8 h. The decomposition of the sample at time t, dₜ, was calculated as:

$${d}_{t} = \frac{{A}_{0} - {A}_{t}}{{A}_{0}} \times 100$$

where A₀ is the absorbance of the solution at time 0, and Aₜ is the absorbance at time t.

The presumed mechanism for the production of radicals from ¹O₂ and squalene involves the formation of singlet oxygen as in normal photodynamic therapy, the attack at a double bond in the squalene by the resulting ¹O₂, the abstraction of hydrogen from a neighboring —CH₂— group, the formation of an alkyl hydroperoxide, and finally the production of free radicals such as HO. and RO.. These radicals may then initiate radical chain reactions and thus generate more radicals.

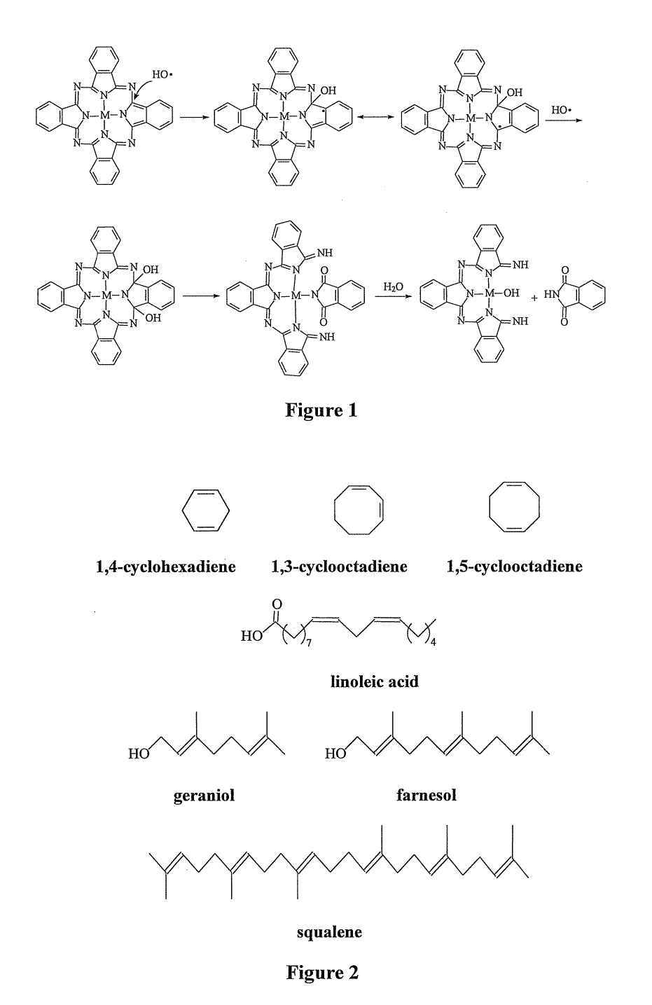

The experimental results provided in Table 1 show the stability of the four Al and Si phthalocyanines decreased significantly in the presence of squalene, air and light. Although attempts to isolate the decomposition products were not made, it appears that one of the main targets of the radicals could be the double bonds in the phthalocyanine pyrrole group. This could lead to destruction of the phthalocyanine macrocycle, as shown in FIG. 1.

TABLE 1
Photodecomposition of Aluminum and Silicon Phthalocyanines in
Toluene (~9 μM) in the Presence of Squalene (1:1000 mole ratio)
under Irradiation with White Light (1.2 kJ/s/m²) in Air.

| | | | radiation | |
|---|---|---|---|---|
| | | time | (kJ/mol × | decomp |
| formula | | (h) | 10⁻⁶) | (%) |
| Pc 285 | AlPcOSiPr₃ | 0 | 0 | 0 |
| | | 2 | 86 | 8 |
| | | 4 | 172 | 16 |
| | | 6 | 258 | 23 |
| | | 8 | 344 | 30 |
| Pc 286 | AlPcOSiHx₃ | 0 | 0 | 0 |
| | | 2 | 86 | 10 |
| | | 4 | 172 | 17 |
| | | 6 | 258 | 23 |
| | | 8 | 344 | 29 |
| Pc 282 | SiPc(OSiPr₃)₂ | 0 | 0 | 0 |
| | | 2 | 86 | 3 |
| | | 4 | 172 | 4 |
| | | 6 | 258 | 6 |
| | | 8 | 344 | 7 |
| Pc 162 | SiPc(OSiHx₃)₂ | 0 | 0 | 0 |
| | | 2 | 86 | 9 |
| | | 4 | 172 | 14 |
| | | 6 | 258 | 20 |
| | | 8 | 344 | 26 |

It is apparent that among the four phthalocyanines studied, the Al phthalocyanines photodecompose most easily. This may be due to steric protection of both sides of the ring of the Si phthalocyanines but only one side of the ring of the Al phthalocyanines. However, the greater photostability of SiPc[OSi(CH₂)₂CH₃)₃]₂ relative to that of SiPc[OSi(CH₂)₅CH₃)₃]₂ is not consistent with this steric protection argument. To further investigate the effect of the axial ligands on the photostability of phthalocyanines, additional experiments were conducted.

### Example 2

### Influence of the Structure of the Axial Ligands on the Photostability of Silicon Phthalocyanines in Presence of Squalene

The decomposition of silicon phthalocyanines can be induced by the combination of squalene, air and light, and the decomposition rate of these phthalocyanines appears to be affected by the nature of the axial ligands. To investigate this axial-ligand effect in more detail, the photostability of a series of silicon phthalocyanines with various axial ligands was studied in the presence of squalene and air. The results show that the structure of the ligands has considerable influence on the stability of these phthalocyanines.

The data provided in Table 2 on the five bis-capped silicon phthalocyanines SiPc[OSi(CH₂)ₙCH₃)₃]₂, where n is 0, 1, 2, 5 and 7 show that their stability increases with increasing size of the axial ligands up to n=2. The trend for the three lighter members of the series is attributed to the greater steric protection provided by larger ligands. The relatively low stability of the two heavier members suggests that processes in addition to radical attack on the ring are involved in their decomposition. Although these processes are not understood, they may involve radical attack on the CH₂ group of the ligands and thus chain shortening,

Steric protection may also explain the low stability of SiPc(OSiH₂CH₃) since its hydridoalkyl ligands do not protect its ring well. However, radical attack on its Si—H bonds may cause further complexities. Similarly, steric protection may explain the low stability of HOSiPcOSi[(CH₂)₅CH₃]₃, because its OH ligand provides little ring protection. It may also explain the low stability of HOSiPcOSi(CH₃)₂(CH₂)₃I, but here radical attack on the C—I bond may add complexity.

TABLE 2
Photodecomposition of Silicon Phthalocyanines in Toluene
(~9 μM) in the Presence of Squalene (1:1000 mole
ratio) under Irradiation with White Light (1.2 kJ/s/m²)

| | | | radiation | |
|---|---|---|---|---|
| | | time | (kJ/mol × | decomp |
| formula | | (h) | 10⁻⁶) | (%) |
| Pc 280 | SiPc(OSiMe₃)₂ | 0 | 0 | 0 |
| | | 2 | 86 | 11 |
| | | 4 | 172 | 19 |
| | | 6 | 258 | 27 |
| | | 8 | 344 | 36 |
| Pc 281 | SiPc(OSiEt₃)₂ | 0 | 0 | 0 |
| | | 2 | 86 | 5 |
| | | 4 | 172 | 9 |
| | | 6 | 258 | 13 |
| | | 8 | 344 | 17 |
| Pc 282 | SiPc(OSiPr₃)₂ | 0 | 0 | 0 |
| | | 2 | 86 | 3 |
| | | 4 | 172 | 4 |
| | | 6 | 258 | 6 |
| | | 8 | 344 | 7 |
| Pc 162 | SiPc(OSiHx₃)₂ | 0 | 0 | 0 |
| | | 2 | 86 | 9 |
| | | 4 | 172 | 14 |
| | | 6 | 258 | 20 |
| | | 8 | 344 | 26 |
| Pc 308 | SiPc(OSiOct₃)₂ | 0 | 0 | 0 |
| | | 2 | 86 | 10 |
| | | 4 | 172 | 15 |
| | | 6 | 258 | 21 |
| | | 8 | 344 | 26 |
| Pc 283 | SiPc(OSiH₂Me)2 | 0 | 0 | 0 |
| | | 2 | 86 | 24 |
| | | 4 | 172 | 44 |
| | | 6 | 258 | 63 |
| | | 8 | 344 | 77 |
| Pc 252 | SiPc(OSiHx₃)(OH) | 0 | 0 | 0 |
| | | 2 | 86 | 16 |
| | | 4 | 172 | 26 |
| | | 6 | 258 | 34 |
| | | 8 | 344 | 42 |
| Pc 58 | SiPc[OSiMe₂(CH₂)₃I](OH) | 0 | 0 | 0 |
| | | 2 | 86 | 12 |
| | | 4 | 172 | 23 |
| | | 6 | 258 | 34 |
| | | 8 | 344 | 44 |
| Pc 4 | SiPc[OSiMe₂(CH₂)₃NMe₂](OH) | 0 | 0 | 0 |
| | | 2 | 86 | 57 |
| | | 4 | 172 | 81 |
| | | 6 | 258 | 92 |
| | | 8 | 344 | 96 |

The low stability of HOSiPcOSi(CH₃)₂(CH₂)₃N(CH₃)₂, Pc 4, relative to the stability of HOSiPcOSi(CH₃)₂(CH₂)₃I and HOSiPcOSi[(CH₂)₅CH₃]₃ suggests that the amino group in Pc 4 plays a role in its photodecomposition. It seems likely that its amino group can be attacked by ¹O₂ and that this results in the generation of free radicals. A mechanism for radical attack on CH₃NRR′-type compounds has been proposed by Baciocchi et al. In this mechanism, an exciplex is formed by charge transfer between the ¹O₂ and the amine, and then the exciplex decomposes to free radicals. Baciocchi et al., Org. Lett. 8, (9), 1783-1786 (2006). Thus, not only can Pc 4 be attacked by radicals from the ¹O₂-squalene pair but also by radicals from the ¹O₂-amino group pair.

### Example 3

### Influence of Replacement of Squalene by Other Alkenes

Since previous work showed that free radicals generated by the attack of ¹O₂ on squalene can induce the decomposition of phthalocyanines, it was of interest to investigate the ability of alkenes in general to cause the photodecomposition of phthalocynines as a function of their structure. This led to studies with 1,4-cyclohexadiene, 1,3-cyclooctadiene, 1,5-cyclooctadiene, linoleic acid, geraniol and farnesol, shown in FIG. 2.

The results of these studies, provided in Table 3, show that the six new alkenes all decrease the photostability of Pc 4, with geraniol and farnesol being the most effective. These latter two are analogues of squalene but only have two or three 2-methyl-2-butene units rather than the six that squalene has. However, although they have fewer double bonds, they show greater effectiveness. This effectiveness appears to be related to the presence of their allylic OH groups. Supporting this conclusion is the ability of ¹O₂ to react with allyl alcohols to form relatively stable hydrogen-bonded complexes. These complexes can then rearrange with the shift of an allylic hydrogen to form hydroperoxides. Clennan et al., Photochem. Photobiol., 82, (5), 1226-1232 (2006). The relatively stability of these complexes probably helps with the generation of the alkyl hydroperoxide and ultimately the production of radicals.

The results also show that 1,5-cyclooctadiene, while effective, is less so than linoleic acid and 1,4-cyclohexadiene. The effectiveness of 1,5-cyclooctadiene probably is due to a reduction in the dissociation energy at its allylic C—H bond caused by pseudo π-conjugation between C═C bond and the allylic C—H bond. In the case of linoleic acid and 1,4-cyclohexadiene, the allylic hydrogens are activated by two π systems and thus they are more reactive (studies show that the rate constant of an allylic hydrogen activated by one it system is ˜10⁻⁶ M⁻¹s⁻¹, while that of an allylic hydrogen activated by two π systems is ˜10⁻³ M⁻¹ s⁻¹) Hamilton et al., Food Chem., 60, (2), 193-199 (1997).

The conjugated alkene 1,3-cyclooctadiene has the lowest efficacy. This may be due to the fact that conjugated dienes tend to react with ¹O₂ via a Diels Alder-like cycloaddition reaction instead of reacting with it to give alkyl hydroperoxides. Supporting this view is the fact that 9c,11c-C18:2-linoleic acid (which is conjugated) does not generate measurable lipid hydroperoxides by radical attack or by photooxidation.

Although geraniol is similar in effectiveness to farnesol at relatively high concentrations, at low concentrations it is less effective, as seen in Tables 3 and 4. The greater effectiveness of farnesol at low concentrations correlates with the high degree of unsaturation in it.

At low concentrations squalene is more effective than geraniol and farnesol, indicating that at low concentrations, the influence of a high degree of unsaturation overcomes the influence of the OH group. The data also show that the photodecomposition of Pc 4 increases as the concentration of the alkenes increases. This is consistent with the greater availability of radicals at the higher concentrations

TABLE 3
Photodecomposition of Pc 4 in Toluene (~9 μM) in the Presence of Alkenes
(1:1000 mole ratio) under Irradiation with White Light (1.2 kJ/s/m²)

| alkene | | time | radiation | decomp |
|---|---|---|---|---|
| name | structure | (h) | (kJ/mol × 10⁻⁶) | (%) |
| — | | 0 | 0 | 0 |
| | | 2 | 86 | 11 |
| | | 4 | 172 | 12 |
| | | 6 | 258 | 12 |
| | | 8 | 344 | 13 |
| 1,3-cyclooctadiene | 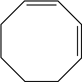 | 0 2 4 6 8 | 0 86 172 258 344 | 0 15 22 30 37 |
| 1,5-cyclooctadiene | 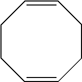 | 0 2 4 6 8 | 0 86 172 258 344 | 0 23 49 69 83 |
| 1,4-cyclohexadiene | 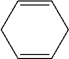 | 0 2 4 6 8 | 0 86 172 258 344 | 0 34 73 88 96 |
| linoleic acid | 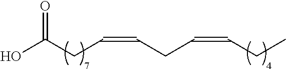 | 0 2 4 6 8 | 0 86 172 258 344 | 0 40 72 89 99 |
| geraniol | 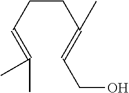 | 0 2 4 6 8 | 0 86 172 258 344 | 0 87 98 99 99 |
| farnesol | 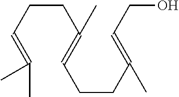 | 0 2 4 6 8 | 0 86 172 258 344 | 0 79 93 99 99 |
| squalene | 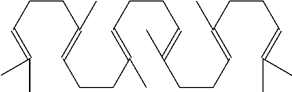 | 0 2 4 6 8 | 0 86 172 258 344 | 0 57 81 92 96 |

TABLE 4
Photodecomposition of Pc 4 in Toluene (~9 μM) in the Presence of Alkenes
(1:100 mole ratio) under Irradiation with White Light (1.2 kJ/s/m²)

| alkene | | time | radiation | decomp |
|---|---|---|---|---|
| name | structure | (h) | (kJ/mol × 10⁻⁶) | (%) |
| geraniol | 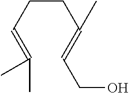 | 0 2 4 6 8 | 0 86 172 258 344 | 0 10 14 21 30 |
| farnesol | 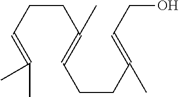 | 0 2 4 6 8 | 0 86 172 258 344 | 0 13 20 38 50 |
| squalene | 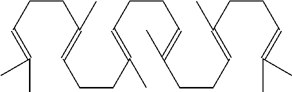 | 0 2 4 6 8 | 0 86 172 258 344 | 0 50 75 87 94 |

### Example 4

### Photostability of Silicon Phthalocyanines with Saturated and Unsaturated Ligands

The previous work showed that silicon phthalocyanines photodecompose under radical attack in the presence of oxygen. Additional experiments were conducted to evaluate the possibility of decreasing the photostability of silicon phthalocyanines in a controlled way by attaching selected radical sources to them. One way to achieve this is to ligate the sources to the silicon. Accordingly, a group of silicon phthalocyanines with unsaturated axial ligands were prepared. The phthalocyanines which were made have unsaturated carboxy ligands, SiPc[OC(O)R]₂; unsaturated alkoxy ligands, SiPc(OR)₂; and unsaturated siloxy ligands, SiPc[OSiRR′R″]₂

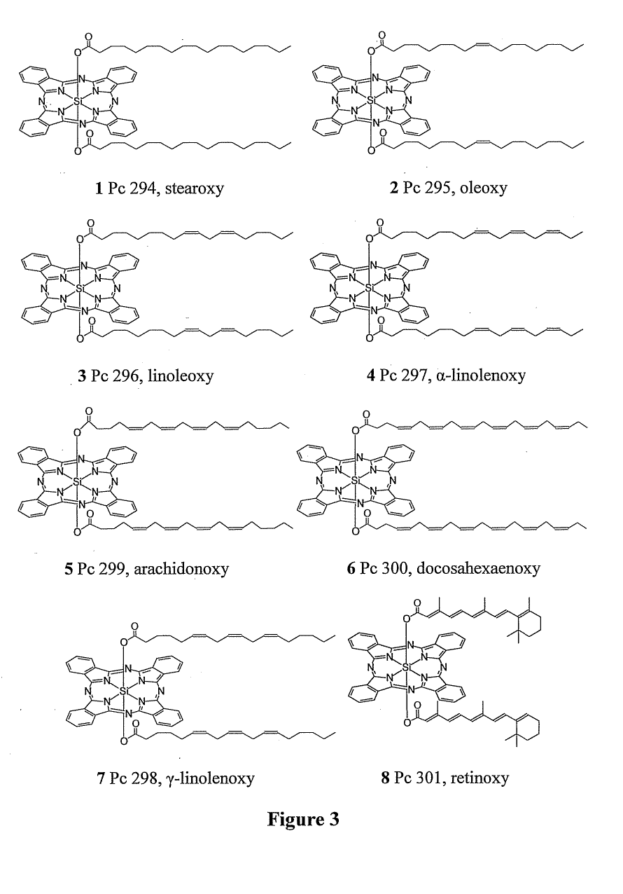

Unsaturated fatty acids with varying degrees of unsaturation can lead to varying rates of photodecomposition in silicon phthalocyanines. It is known in the art that carboxylic acids can bond to the central silicon of silicon phthalocyanines through their carboxy groups. Thus, it was concluded that a series of unsaturated carboxysilicon phthalocyanines could be prepared which would have varying degrees of photostability. This led to the preparation and study of eight carboxysilicon phthalocyanines, shown in FIG. 3.

The UV-vis absorption spectra of these phthalocyanines show that their long wavelength absorptions are at around 684 nm in toluene. These absorptions are red-shifted by about 10 nm by relative to those of alkoxysilicon phthalocyanines such as SiPc(OC₈H₁₇)₂. This red shift is attributed to the electron withdrawing ability of the carboxy ligands.

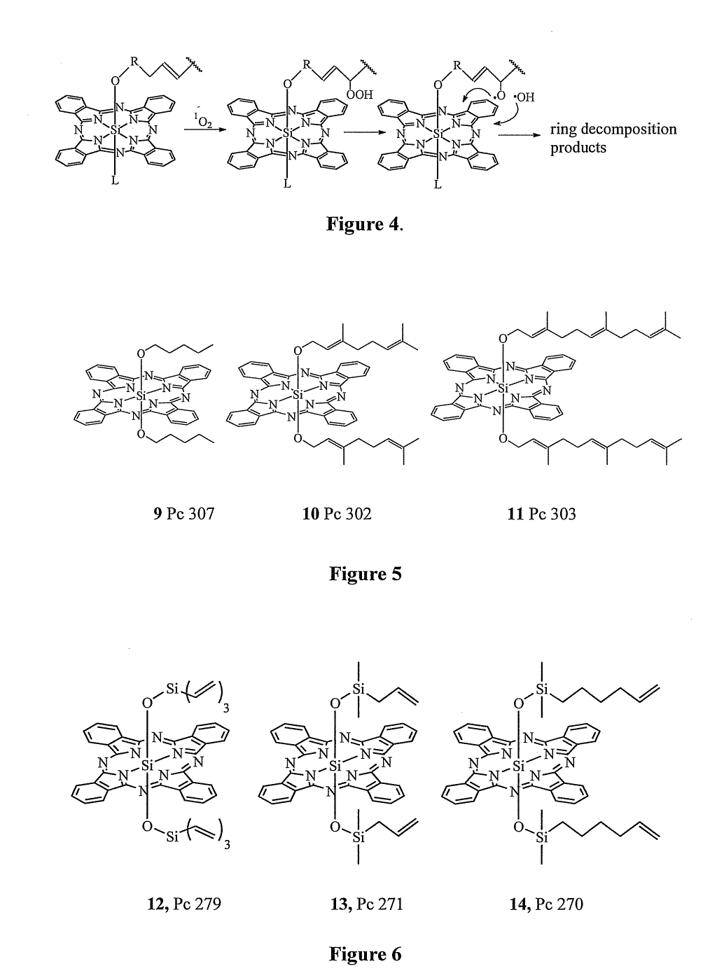

For phthalocyanines 1-5, the Q and Soret bands decrease over time under irradiation with white light, and no new bands appear. Based on a knowledge of the photostability of silicon and aluminum phthalocyanines in presence of alkenes, the process leading to this decomposition appears to involve: the formation of ¹O₂ as in normal photodynamic therapy, the attack at a double bond of the axial ligands by ¹O₂, the production of free radicals, and finally the decomposition of the phthalocyanine by radical attack, as shown in FIG. 4.

In the case of bis(docosahexaenoxy)silicon phthalocyanine, 6, a band appears at around 672 while the Q band (at 684 nm) decreases during the first 8 hours of irradiation. After this, the 672 nm band also decreases. This result indicates that a second process also takes place along with the normal degradation of the phthalocyanine ring. Although this putative process is not understood, it might involve the displacement of one of the docosahexaenoxy groups by a OH group. The process appears to be induced by light and may be catalyzed by a high concentration of free radicals since it has not been observed in the first 8 hours with the carboxysilicon phthalocyanines containing fewer double bonds.

The order of photostability of all but the bis(γ-linolenoxy)silicon and bis(retinoxy)silicon phthalocyanines is: stearoxy>oleoxy>linoleoxy>α-inolenoxy>arachidonoxy>docosahexaenoxy, as shown in Table 5. This correlates with the number of double bonds in the ligands (0, 1, 2, 3, 4, 6, respectively). The lower photostability of carboxysilicon phthalocyanines with more double bonds is attributed to the greater susceptibility to ¹O₂ attack of these ligands with greater unsaturation.

Bis(γ-linolenoxy)silicon phthalocyanine decomposes approximately twice as fast as bis(α-linolenoxy)silicon phthalocyanine, although the two isomers have the same degree of unsaturation. This is attributed to the shorter paths which the ¹O₂ and free radicals have to traverse in the γ isomer.

TABLE 5
Photodecomposition of Carboxysilicon Phthalocyanines in Toluene (~9 μM) under
Irradiation with White Light (1.2 kJ/s/m²)

| | | | | radiation | de- |
|---|---|---|---|---|---|
| | ligand | | time | (kJ/mol × | comp |
| | name | structure | (h) | 10⁻⁶) | (%) |
| Pc 294 1 | stearoxy | 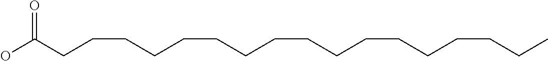 | 0 2 4 6 8 | 0 86 172 258 344 | 0 1 1 1 2 |
| Pc 295 2 | oleic acid | 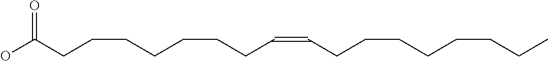 | 0 2 4 6 8 | 0 86 172 258 344 | 0 2 3 3 4 |
| Pc 296 3 | lino- leoxy | 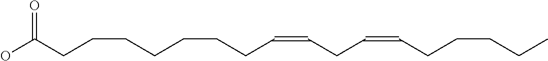 | 0 2 4 6 8 | 0 86 172 258 344 | 0 2 3 4 5 |
| Pc 297 4 | α-lino- lenoxy | 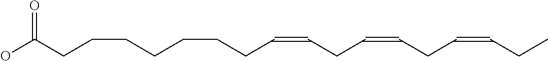 | 0 2 4 6 8 | 0 86 172 258 344 | 0 3 5 6 9 |
| Pc 298 5 | arachid- onoxy | 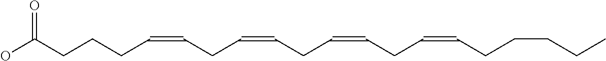 | 0 2 4 6 8 | 0 86 172 258 344 | 0 12 21 27 33 |
| Pc 300 6 | docosa- hexa- enoxy | 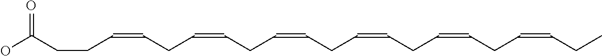 | 0 2 4 6 8 | 0 86 172 258 344 | 0 16 34 46 56 |
| Pc 299 7 | γ-lino- lenoxy | 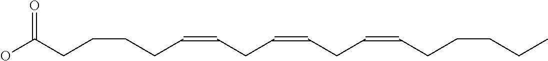 | 0 2 4 6 8 | 0 86 172 258 344 | 0 7 12 14 17 |
| Pc 301 8 | retinoxy | 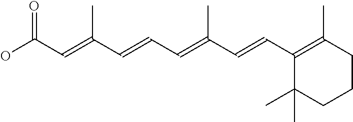 | 0 2 4 6 8 | 0 86 172 258 344 | 0 19 25 28 32 |

Bis(retinoxy)silicon phthalocyanine, 8, which has 5 conjugated double bonds in each ligand, and bis(arachidonoxy)silicon phthalocyanine, 5, which has 4 unconjugated double bonds in each ligand, show nearly the same amount of photodecomposition after 8 hours, as shown in Table 6. The higher stability of the retinoxysilicon phthalocyanine is attributed to the conjugation of the double bonds in its ligands. This gives the ligands the potential to be both singlet oxygen quenchers and free radical scavengers by energy transfer and through the formation of low-activity carbon-centered radicals, as is the case with β-carotene.

TABLE 6
Photodecomposition of Arachidonoxysilicon and Retinoxysilicon
Phthalocyanines in Toluene (~9 μM) under Irradiation with White Light (1.2 kJ/s/m²)

| | ligand | | | radiation | de- |
|---|---|---|---|---|---|
| | | | time | (kJ/mol × | comp |
| | name | structure | (h) | 10⁻⁶) | (%) |
| Pc 299 5 | arachidonoxy | 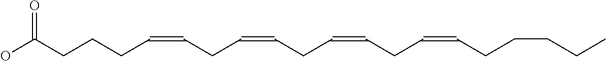 | 0 2 4 6 8 | 0 86 172 258 344 | 0 12 21 27 33 |
| Pc 301 8 | retinoxy | 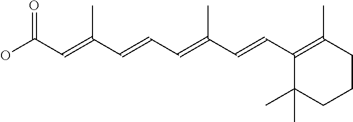 | 0 2 4 6 8 | 0 86 172 258 344 | 0 19 25 28 32 |

The photostability of the alkoxysilicon phthalocyanines, pentoxysilicon phthalocyanine, geranoxysilicon phthalocyanine and farnesoxysilicon phthalocyanine was studied. The structures of these compounds are shown in FIG. 5. All of these compounds are stable in the dark, but the two with unsaturated ligands decompose under white light, as can be seen in Table 7. The photodecomposition of the two unsaturated alkoxysilicon phthalocyanines is more rapid than that of the unsaturated carboxysilicon phthalocyanines 3, the linoxy and 7, the γ-linolenoxy, under comparable conditions. This is ascribed mostly to the greater proximity of their ligand double bonds to the phthalocyanine ring.

TABLE 7
Photodecomposition of Alkoxysilicon Phthalocyanines in Toluene (~9 μM)
under Irradiation with White Light (1.2 kJ/s/m²)

| | ligand | | time | radiation | decomp |
|---|---|---|---|---|---|
| | name | structure | (h) | (kJ/mol × 10⁻⁶) | (%) |
| Pc 307 9 | pentoxy | 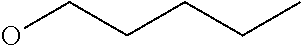 | 0 2 4 6 8 | 0 86 172 258 344 | 0 23 55 80 90 |
| Pc 302 10 | geranoxy | 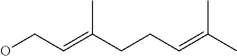 | 0 2 4 6 8 | 0 86 172 258 344 | 0 19 60 74 81 |
| Pc 303 11 | farnesoxy | 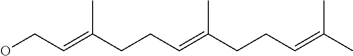 | 0 2 4 6 8 | 0 86 172 258 344 | 0 1 2 2 2 |

### Example 6

### Photostability of Siloxysilicon Phthalocyanines in White Light

Three unsaturated siloxysilicon phthalocyanines: 12, SiPc[OSi(CH═CH₂)₃]₂, Pc 279, 13, SiPc[OSi(CH₃)₂CH₂CH═CH₂]₂, Pc 271, and 14, SiPc[OSi(CH₃)₂(CH₂)₄CH═CH₂]₂, Pc 270 were synthesized and their photodegradation was studied. The structures of these compounds are shown in FIG. 6. The results show that the vinyl phthalocyanine is quite stable, remaining unchanged during 8 hours of irradiation. This is attributed to its lack of allylic hydrogens, since without these it cannot give rise to an alkyl hydroperoxide through reaction with ¹O₂ and thus generate radicals.

The results, provided in Table 8, also show that SiPc[OSi(CH₃)₂CH₂CH═CH₂]₂, Pc 271, decomposes significantly more rapidly than SiPc[OSi(CH₃)₂(CH₂)₄CH═CH₂]₂, Pc 270, although they each have one double bond per ligand. The lower photostability of SiPc[OSi(CH₃)₂CH₂CH═CH₂]₂ is ascribed to the greater proximity of its ligand double bonds to the phthalocyanine ring.

TABLE 8
Photodecomposition of Unsaturated Siloxysilicon Phthalocyanines in
Toluene (~9 μM) under Irradiation with White Light (1.2 kJ/s/m²)

| | | | radiation | de- |
|---|---|---|---|---|
| | | time | (kJ/mol × | comp |
| | ligand | (h) | 10⁻⁶) | (%) |
| Pc 279 12 | 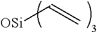 | 0 2 4 6 8 | 0 86 172 258 344 | 0 0 0 0 0 |
| Pc 271 13 | 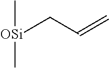 | 0 2 4 6 8 | 0 86 172 258 344 | 0 6 10 14 16 |
| Pc 270 14 | 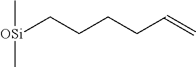 | 0 2 4 6 8 | 0 86 172 258 344 | 0 1 2 2 3 |

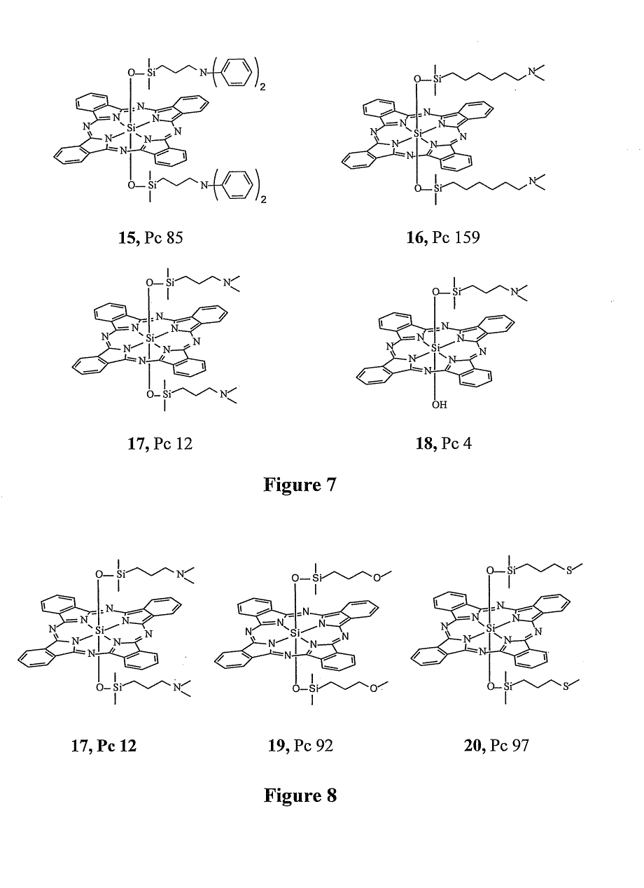

The susceptibility of the aminosiloxysilicon phthalocyanine, 18, HOSiPcOSi(CH₃)₂(CH₂)₃N(CH₃)₂, Pc 4, to photodecomposition has been described. Its susceptibility has been attributed to self-generated radicals. To further investigate the photodecomposition of aminosiloxysilicon phthalocyanines, Pc 4 and three other aminosiloxysilicon phthalocyanines, SiPc[OSi(CH₃)₂(CH₂)₃N(CH₃)₂]₂, Pc 12, SiPc[OSi(CH₃)₂(CH₂)₃N(C₆H₅)₂]₂, Pc 159 and SiPc[OSi(CH₃)₂(CH₂)₆N(CH₃)₂]₂, Pc 85, were studied. The structures of these compounds are shown in FIG. 7.

The results of these studies, provided in Table 9, show that the phenylamino compound SiPc[OSi(CH₃)₂(CH₂)₃N(C₆H₅)₂]₂, Pc 159, is quite stable under light. Its substantial stability is attributed to both the steric protection provided by the phenyl groups to the amino nitrogen, and to a reduction in electron density at the amino nitrogen caused by these groups (both reduce the susceptibility of the nitrogen to ¹O₂ attack and radical production). The greater photostability of SiPc[OSi(CH₃)₂(CH₂)₆N(CH₃)₂]₂, Pc 85, compared to that of SiPc[OSi(CH₃)₂(CH₂)₃N(CH₃)₂]₂, Pc 12, is attributed to the greater distance between its amino groups and the phthalocyanine ring.

TABLE 9
Photodecomposition of Aminosiloxysilicon Phthalocyanines in Toluene (~9
μM) under Irradiation with White Light (1.2 kJ/s/m²)

| | | time | radiation | decomp |
|---|---|---|---|---|
| | ligand | (h) | (kJ/mol × 10⁻⁶) | (%) |
| Pc 159 15 | 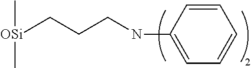 | 0 2 4 6 8 | 0 86 172 258 344 | 0 0 0 0 1 |
| Pc 85 16 | 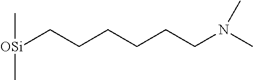 | 0 2 4 6 8 | 0 86 172 258 344 | 0 5 5 5 5 |
| Pc 12 17 | 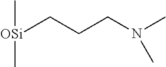 | 0 2 4 6 8 | 0 86 172 258 344 | 0 19 20 21 21 |

Comparison of the photostability of HOSiPc[OSi(CH₃)₂(CH₂)₃N(CH₃)₂], Pc 4, and SiPc[OSi(CH₃)₂(CH₂)₃N(CH₃)₂]₂, Pc 12, shows that Pc 4 is more stable. See Table 10. This is attributed to the presence of only one amino group in Pc 4.

TABLE 10
Photodecomposition of Mono- and Bis(aminosiloxy)silicon
Phthalocyanines in Toluene(~9 μM) under Irradiation
with White Light (1.2 kJ/s/m²)

| | | | radiation | |
|---|---|---|---|---|
| | | time | (kJ/mol × | decomp |
| | | (h) | 10⁻⁶) | (%) |
| Pc 12 | 17 | 0 | 0 | 0 |
| | | 2 | 86 | 18 |
| | | 4 | 172 | 19 |
| | | 6 | 258 | 21 |
| | | 8 | 344 | 21 |
| Pc 4 | 18 | 0 | 0 | 0 |
| | | 2 | 86 | 11 |
| | | 4 | 172 | 12 |
| | | 6 | 258 | 12 |
| | | 8 | 344 | 13 |

### Example 7

### Photostability of a Methoxysiloxysilicon Phthalocyanine and a Thiomethoxysiloxysilicon Phthalocyanine

Since alkylaminosiloxysilicon phthalocyanines have low photostability, it was of interest to examine the photostability of phthalocyanines bearing other heteroatoms. With this in mind a comparative study of Pc 12 and its methoxy and thiomethoxy analogues was carried out. These structures are shown in FIG. 8, and the results are shown in Table 11. The data for SiPc[OSi(CH₃)₂(CH₂)₃OCH₃]₂, Pc 92, show that its methoxy ligands are much less effective than the amino ligands of Pc 12 in causing photodecomposition of the phthalocyanine ring. This is attributed to the less basic character of its hetero atoms because this lowered basicity could lead to them being less efficient in generating radicals in the presence of ¹O₂. The results for SiPc[OSi(CH₃)₂(CH₂)₃SCH₃]₂, Pc 97, show that it is more sensitive to photodegradation than Pc 92. This probably arises because its hetero atoms are more basic. The mechanism of the decomposition may involve reactions in which ¹O₂ first reacts with the thiomethoxy group to form a hydroperoxysulfonium ylide, and then the ylide decomposes to give a thiomethyl radical.

TABLE 11
Photodecomposition of Aminosiloxysilicon, Methoxysiloxysilicon and
Thiomethoxysiloxysilicon Phthalocyanines in Toluene (~9 μM) under Irradiation with White
Light (1.2 kJ/s/m²)

| | | time | radiation | decomp |
|---|---|---|---|---|
| | ligand | (h) | (kJ/mol × 10⁻⁶) | (%) |
| Pc 12 17 | 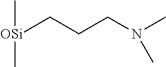 | 0 2 4 6 8 | 0 86 172 258 344 | 0 18 19 21 21 |
| Pc 92 19 | 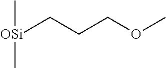 | 0 2 4 6 8 | 0 86 172 258 344 | 0 1 1 1 1 |
| Pc 97 20 | 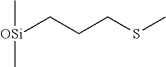 | 0 2 4 6 8 | 0 86 172 258 344 | 0 41 42 43 44 |

### Example 8

### Photostability of Saturated and Unsaturated Pc 12 Salts

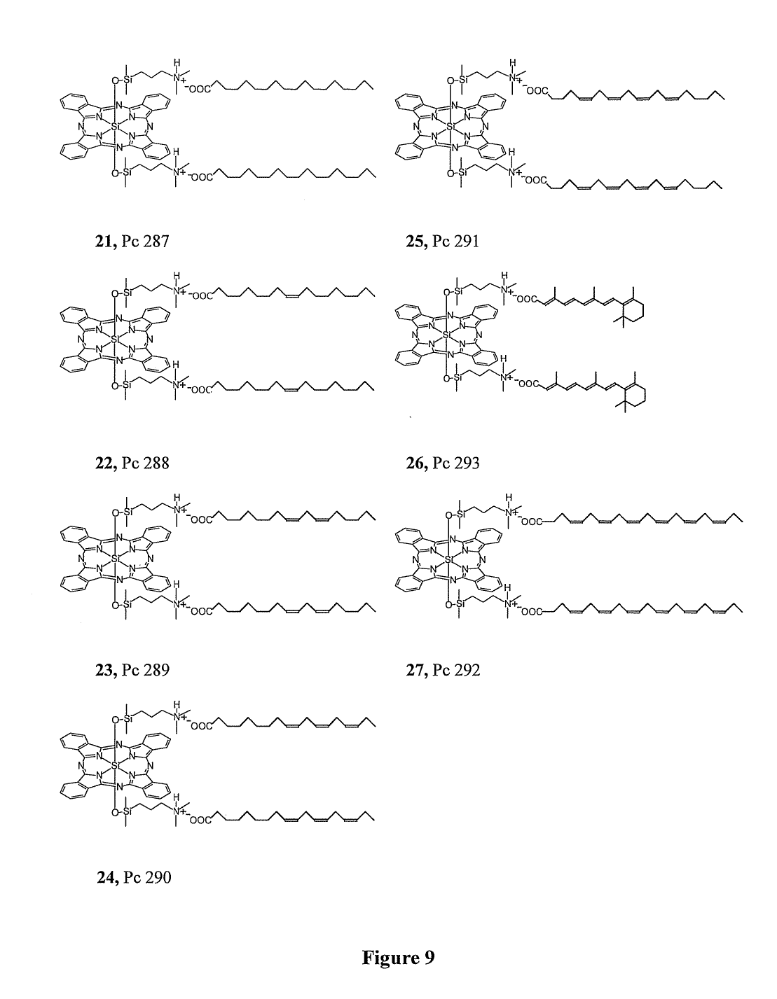

The basic character of the amino groups in Pc 12 opens the possibility of the synthesis phthalocyanine salts having unsaturated carboxylic acid anions, and thus a means of controlling the stability of silicon phthalocyanines through the attachment of radical sources. This led to the synthesis of a set of seven saturated and unsaturated carboxylic acid salts of Pc 12: the stearate, 21; oleate, 22; linoleate, 23; linolenate, 24; arachidonate, 25; retinoate, 26; and docosahexaenoate, 27. The structures of these compounds are shown in FIG. 9. Ultimately four of these salts: the stearate, the linoleate, the arachidonate and the docosahexaenoate were chosen as example salts for photostability studies.

The results of these studies, provided in Table 12, show that all four salts have stabilities similar to that of Pc 12. A possible explanation for this similarity is that the amino salt groups in these compounds are closer to the ring than the double bonds, and thus are more influential.

TABLE 12
Photodecomposition of Saturated and Unsaturated Pc 12 Salts in Toluene (~9 μM)
under Irradiation with White Light (1.2 kJ/s/m²)

| | | | | radiation | de- |
|---|---|---|---|---|---|
| | parent acid | | time | (kJ/mol × | comp |
| | name | structure | (h) | 10⁻⁶) | (%) |
| Pc 12 17 | — | — | 0 | 0 | 0 |
| | | | 2 | 86 | 18 |
| | | | 4 | 172 | 19 |
| | | | 6 | 258 | 21 |
| | | | 8 | 344 | 21 |
| Pc 287 21 | stearic | 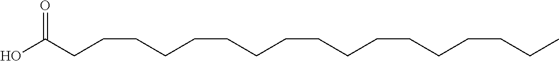 | 0 2 4 6 8 | 0 86 172 258 344 | 0 19 20 21 21 |
| Pc 289 23 | linoleic | 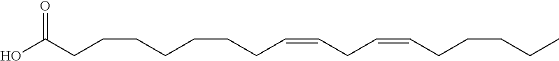 | 0 2 4 6 8 | 0 86 172 258 344 | 0 18 19 20 21 |
| Pc 291 25 | arachidonic | 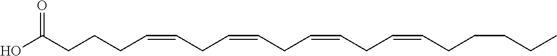 | 0 2 4 6 8 | 0 86 172 258 344 | 0 18 19 20 20 |
| Pc 292 27 | docosahexa- enoate | 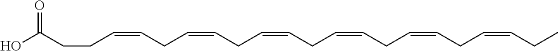 | 0 2 4 6 8 | 0 86 172 258 344 | 0 20 21 22 22 |

### Example 9

### Photostability of Aluminum Phthalocyanines in White Light

As an initial step in the investigation of the photostability of other metal phthalocyanines caused by self-generated radicals, a comparative study of the stability of AlPcOSi(CH₃)₂(CH₂)₃N(CH₃)₂ Pc 1, 43 and Pc 4 was made. The structure of compound 43 is shown below:

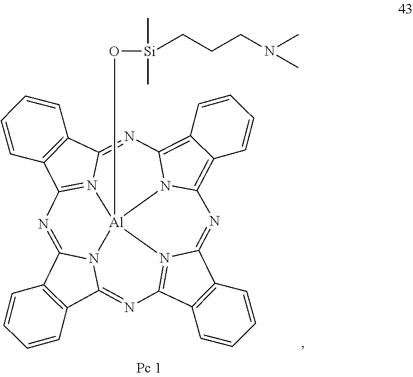

The data provided in Table 13 show that Pc 1, the direct aluminum analogue of Pc 4, has low photostability. As with Pc 4, the amino group can react with ¹O₂ to generate radicals and cause decomposition of the ring. The mechanism for this reaction is likely similar to that seen with aminosilxysilicon phthalocyanines. The data also show that Pc 1 decomposes faster than Pc 4 under comparable conditions. This can be attributed to differences between the aluminum and silicon phthalocyanine rings.

TABLE 13
Photodecomposition of Pc 1 and Pc 4 in Toluene (~9 μM) under
Irradiation with White Light (1.2 kJ/s/m²)

| | | | radiation | de- |
|---|---|---|---|---|
| | | time | (kJ/mol × | comp |
| | structure | (h) | 10⁻⁶) | (%) |
| Pc 1 43 | 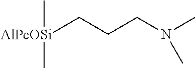 | 0 2 4 6 8 | 0 86 172 258 344 | 0 27 39 46 49 |
| Pc 4 18 | 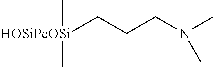 | 0 2 4 6 8 | 0 86 172 258 344 | 0 11 12 12 13 |

### Example 10

### Synthesis of Phthalocyanines

The SiPcCl₂ used was primarily purchased from an organometallics supplier (Gelest, Tullytown, Pa.). The organosilicon reagents were purchased from the same supplier. The other reagents and solvents were purchased from chemical vendors (e.g., Aldrich, Milwaukee, Wis.; Fisher Scientific, Pittsburgh, Pa.; and Acros Organics, Morris Plains, N.J.). Most of the chemicals were of reagent grade. Compounds which are temperature sensitive, such as inosinic acid, were stored in a refrigerator. Pyruvic acid was purified and dried by partial distillation. The preparation of numerous phthalocyanine derivatives is described in international patent publication WO 2005/099689, the disclosure of which is incorporated by reference herein.

### Carboxysilicon Phthalocyanines

Compound 1: SiPc[OC(O)(CH₂)₁₆CH₃]₂, Pc 294.

Under Ar, a mixture of SiPc(OH)₂ (13 mg, 0.020 mmol) and stearic acid (105 mg, 0.370 mmol) was stirred at 130° C. for 3 h, diluted with CH₂Cl₂ (5 mL), filtered, and evaporated to dryness by rotary evaporation (room temperature). The solid was chromatographed (neutral Al₂O₃ III, CH₂Cl₂), washed (CH₃CN), vacuum dried (room temperature) and weighed (11 mg, 0.010 mmol, 50%). UV-vis (toluene) λₘₐₓ, nm (log ε): 683 (5.4). NMR (CDCl₃): δ 9.65 (m, 8H, 1,4-Pc H), 8.37 (m, 8H, 2,3-Pc H), 1.40-1.10 (m, 36H, OC(O)(CH₂)₇(CH₂)₉), 1.00 (m, 4H, OC(O)(CH₂)₆CH₂), 0.87 (t, 6H, OC(O)(CH₂)₇(CH₂)₉CH₃), 0.80 (m, 4H, OC(O)(CH₂)₅CH₂), 0.50 (m, 4H, OC(O)(CH₂)₄CH₂), 0.01 (m, 4H, OC(O)(CH₂)₃CH₂), −0.65 (t, 4H, OC(O)CH₂), −0.79 (m, 4H, OC(O)(CH₂)₂CH₂), −1.00 (m, 4H, OC(O)CH₂CH₂). HRMS-ESI (m/z): [M+Na]⁺ calcd for M as C₆₈H₈₆N₈O₄Si, 1129.6439. found, 1129.6480.

Compound 1 is a blue solid. It is soluble in CH₂Cl₂, dimethylformamide, pyridine and toluene, slightly soluble in hexanes, and insoluble in methanol. The structures of Compounds 1-8 are shown in FIG. 3.

Compound 2: SiPc[OC(O)(CH₂)₇CH═CH(CH₂)₇CH₃]₂, Pc 295.

Under Ar, a mixture of SiPc(OH)₂ (20 mg, 0.030 mmol) and oleic acid (100 mg, 0.370 mmol) was stirred at 130° C. for 3 h, diluted with CH₂Cl₂ (5 mL), filtered, and evaporated to dryness by rotary evaporation (room temperature). The solid was chromatographed (neutral Al₂O₃ III, CH₂Cl₂), washed (CH₃CN), vacuum dried (room temperature) and weighed (15 mg, 0.014 mmol, 45%). UV-vis (toluene) λₘₐₓ, nm (log ε): 683 (5.4). NMR (CDCl₃): δ 9.65 (m, 8H, 1,4-Pc H), 8.37 (m, 8H, 2,3-Pc H), 5.30 (m, 4H, OC(O)(CH₂)₇CH═CH), 1.95 (m, 4H, OC(O)(CH₂)₇CH═CHCH₂), 1.80 (m, 4H, OC(O)(CH₂)₆CH₂), 1.40-1.20 (m, 24H, OC(O)(CH₂)₅CH₂CH₂CH═CHCH₂(CH₂)₆), 0.90-0.80 (m, 10H, OC(O)(CH₂)₅CH₂CH₂CH═CH(CH₂)₇CH₃), 0.55 (m, 4H, OC(O)(CH₂)₄CH₂), 0.03 (m, 4H, OC(O)(CH₂)₃CH₂), −0.65 (t, 4H, OC(O)CH₂), −0.80 (m, 4H, OC(O)(CH₂)₂CH₂), −1.00 (m, 4H, OC(O)CH₂CH₂).

Compound 2 is a blue solid. It is soluble in CH₂Cl₂, dimethylformamide, pyridine and toluene, slightly soluble in hexanes, and insoluble in methanol.

Compound 3: SiPc[OC(O)(CH₂)₆(CH₂CH═CH)₂(CH₂)₄CH₃]₂, Pc 296.

Under Ar, a mixture of SiPc(OH)₂ (20 mg, 0.030 mmol) and linoleic acid (0.10 mL, 0.32 mmol) was stirred at 130° C. for 3 h, diluted with CH₂Cl₂ (5 mL), filtered, and evaporated to dryness by rotary evaporation (room temperature). The solid was chromatographed (neutral Al₂O₃ III, CH₂Cl₂), washed (CH₃CN), vacuum dried (room temperature) and weighed (17 mg, 0.015 mmol, 50%). UV-vis (toluene) λₘₐₓ, nm (log ε): 683 (5.4). NMR (CDCl₃): δ 9.65 (m, 8H, 1,4-Pc H), 8.35 (m, 8H, 2,3-Pc H), 5.30 (m, 8H, OC(O)(CH₂)₆(CH₂CH═CH)₂), 2.70 (m, 4H, OC(O)(CH₂)₆CH₂CH═CHCH₂), 2.00 (m, 4H, OC(O)(CH₂)₆(CH₂CH═CH)₂CH₂), 1.80 (m, 4H, OC(O)(CH₂)₆CH₂CH═CH), 1.40-1.20 (m, 12H, CH₃(CH₂)₃), 0.90-0.80 (m, 10H, OC(O)(CH₂)₅CH₂(CH₂CH═CH)₂(CH₂)₄CH₃), 0.55 (m, 4H, OC(O)(CH₂)₄CH₂), 0.03 (m, 4H, OC(O)(CH₂)₃CH₂), −0.65 (t, 4H, OC(O)CH₂), −0.79 (m, 4H, OC(O)(CH₂)₂CH₂), −1.00 (m, 4H, OC(O)CH₂CH₂).

Compound 3 is a blue solid. It is soluble in CH₂Cl₂, dimethylformamide, pyridine and toluene, slightly soluble in hexanes, and insoluble in methanol.

Compound 4: SiPc[OC(O)(CH₂)₆(CH₂CH═CH)₃CH₂CH₃]_{z}, Pc 297.

Under Ar, a mixture of SiPc(OH)₂ (20 mg, 0.030 mmol) and linolenic acid (0.050 mL, 0.16 mmol) was stirred at 130° C. for 3 h, diluted with CH₂Cl₂ (5 mL), filtered, and evaporated to dryness by rotary evaporation (room temperature). The solid was chromatographed (neutral Al₂O₃ III, CH₂Cl₂), washed (CH₃CN), vacuum dried (room temperature) and weighed (12 mg, 0.011 mmol, 38%). UV-vis (toluene) λₘₐₓ, nm (log ε): 683 (5.4). NMR (CDCl₃): δ 9.65 (m, 8H, 1,4-Pc H), 8.35 (m, 8H, 2,3-Pc H), 5.30 (m, 12H, OC(O)(CH₂)₆(CH₂CH═CH)₃), 2.75 (m, 8H, OC(O)(CH₂)₆CH₂CH═CHCH₂CH═CHCH₂), 2.00 (m, 4H, OC(O)(CH₂)₆(CH₂CH═CH)₃CH₂), 1.80 (m, 4H, OC(O)(CH₂)₆CH₂), 0.90 (m, 10H, OC(O)(CH₂)₅CH₂(CH═CHCH₂)₃CH₂CH₃), 0.55 (m, 4H, OC(O)(CH₂)₄CH₂), 0.03 (m, 4H, OC(O)(CH₂)₃CH₂), −0.65 (t, 4H, OC(O)CH₂), −0.79 (m, 4H, OC(O)(CH₂)₂CH₂), −1.00 (m, 4H, OC(O)CH₂CH₂).

Compound 4 is a blue solid. It is soluble in CH₂Cl₂, dimethylformamide, pyridine and toluene, slightly soluble in hexanes, and insoluble in methanol.

Compound 5: SiPc[OC(O)(CH₂)₂(CH₂CH═CH)₄(CH₂)₄CH₃]₂, Pc 299.

Under Ar, a mixture of SiPc(OH)₂ (20 mg, 0.030 mmol) and arachidonic acid (0.030 mL, 0.11 mmol) was stirred at 130° C. for 4 h, diluted with CH₂Cl₂ (5 mL), filtered, and evaporated to dryness by rotary evaporation (room temperature). The solid was chromatographed (neutral Al₂O₃ (III), CH₂Cl₂), washed (CH₃CN), vacuum dried (room temperature) and weighed (8 mg, 0.0069 mmol, 23%). UV-vis (toluene) λₘₐₓ, nm (log ε): 684 (5.4). NMR (CDCl₃): δ 9.65 (m, 8H, 1,4-Pc H), 8.35 (m, 8H, 2,3-Pc H), 5.30-5.10 (m, 10H, OC(O)(CH₂)₂CH₂CH═CHCH₂CH═CH(CH₂CH═CH)₂), 4.85 (m, 2H, OC(O)(CH₂)₂CH₂CH═CHCH₂CH), 4.65 (m, 2H, OC(O)(CH₂)₂CH₂CH═CH), 4.05 (m, 2H, OC(O)(CH₂)₂CH₂CH), 2.62 (t, 4H, OC(O)(CH₂)₂(CH₂CH═CH)₃CH₂), 2.53 (t, 4H, OC(O)(CH₂)₂(CH₂CH═CH)₂CH₂), 2.00 (t, 4H, OC(O)(CH₂)₃CH═CHCH₂), 1.95 (m, 4H, OC(O)(CH₂)₂(CH₂CH═CH)₄CH₂), 1.30-1.15 (m, 12H, OC(O)(CH₂)₂(CH₂CH═CH)₄CH₂(CH₂)₃), 0.80 (m, 6H, CH₃), 0.25 (m, 4H, OC(O)(CH₂)₂CH₂), −0.67 (t, 4H, OC(O)CH₂), −0.80 (m, 4H, OC(O)CH₂CH₂). HRMS-ESI (m/z): [M+Na]⁺ calcd for M as C₇₂H₇₈N₈O₄Si, 1169.5813. found, 1169.5855.

Compound 5 is a blue solid. It is soluble in CH₂Cl₂, dimethylformamide, pyridine and toluene, slightly soluble in hexanes, and insoluble in methanol.

Compound 6: SiPc[OC(O)CH₂(CH₂CH═CH)₆CH₂CH₃]₂, Pc 300.

Under Ar, a mixture of SiPc(OH)₂ (20 mg, 0.030 mmol) and cis-4,7,10,13,16,19-docosahexaenoic acid (DHA) (0.030 mL, 0.10 mmol) was stirred at 130° C. for 4 h, diluted with CH₂Cl₂ (5 mL), filtered, and evaporated to dryness by rotary evaporation (room temperature). The solid was chromatographed (neutral Al₂O₃ III, CH₂Cl₂), washed (CH₃CN), vacuum dried (room temperature) and weighed (10 mg, 0.0084 mmol, 28%). UV-vis (toluene) λₘₐₓ, nm (log ε): 684 (5.4). NMR (CDCl₃): δ 9.65 (m, 8H, 1,4-Pc H), 8.35 (m, 8H, 2,3-Pc H), 5.40-5.20 (m, 14H, OC(O)CH₂(CH₂CH═CH)₂CH₂CH═CH(CH₂CH═CH)₃), 5.15 (m, 2H, OC(O)CH₂(CH₂CH═CH)₂CH₂CH), 5.02 (m, 2H, OC(O)CH₂CH₂CH═CHCH₂CH═CH), 4.65 (m, 2H, OC(O)CH₂CH₂CH═CHCH₂CH), 4.35 (m, 2H, OC(O)CH₂CH₂CH═CH), 3.30 (m, 2H, OC(O)CH₂CH₂CH), 2.75 (m, 8H, OC(O)CH₂(CH₂CH═CH)₄CH₂CH═CHCH₂), 2.65 (t, 4H, OC(O)CH₂(CH₂CH═CH)₃CH₂), 2.40 (t, 4H, OC(O)CH₂(CH₂CH═CH)₂CH₂), 2.00 (t, 4H, OC(O)CH₂(CH₂CH═CH)₆CH₂), 1.75 (t, 4H, OC(O)CH₂CH₂CH═CHCH₂), 1.00 (m, 6H, OC(O)CH₂(CH₂CH═CH)₆CH₂CH₃), −0.05 (t, 4H, OC(O)CH₂CH₂), −0.65 (t, 4H, OC(O)CH₂). HRMS-ESI (m/z): [M+Na]⁺ calcd for M as C₇₆H₇₈N₈O₄Si, 1217.5813. found, 1217.5855.

Compound 6 is a blue solid. It is soluble in CH₂Cl₂, dimethylformamide, pyridine and toluene, slightly soluble in hexanes, and insoluble in methanol.

Compound 7: SiPc[OC(O)(CH₂)₃(CH₂CH═CH)₃(CH₂)₄CH₃]₂, Pc 298.

Under Ar, a mixture of SiPc(OH)₂ (20 mg, 0.030 mmol) and γ-linolenic acid (0.10 mL, 0.16 mmol) was stirred at 130° C. for 3 h, diluted with CH₂Cl₂ (5 mL), filtered, and evaporated to dryness by rotary evaporation (room temperature). The solid was chromatographed (neutral Al₂O₃ III, CH₂Cl₂), washed (CH₃CN), vacuum dried (room temperature) and weighed (14 mg, 0.013 mmol, 43%). UV-vis (toluene) λₘₐₓ, nm (log ε): 684 (5.4). NMR (CDCl₃): δ 9.65 (m, 8H, 1,4-Pc H), 8.35 (m, 8H, 2,3-Pc H), 5.30 (m, 2H, (O)(CH₂)₃(CH₂CH═CH)₂CH₂CH═CH), 5.20 (m, 4H, (O)(CH₂)₃CH₂CH═CHCH₂CH═CHCH₂CH), 5.05 (m, 2H, (O)(CH₂)₃CH₂CH═CHCH₂CH), 4.93 (m, 2H, OC(O)(CH₂)₄CH═CH), 4.55 (m, 2H, OC(O)(CH₂)₄CH), 2.60 (t, 4H, (O)(CH₂)₃(CH₂CH═CH)₂CH₂), 2.30 (t, 4H, OC(O)(CH₂)₄CH═CHCH₂), 1.95 (m, 4H, (O)(CH₂)₃(CH₂CH═CH)₃CH₂), 1.30-1.15 (m, 12H, (O)(CH₂)₃(CH₂CH═CH)₃CH₂(CH₂)₃), 0.90 (m, 4H, OC(O)(CH₂)₃CH₂), 0.80 (t, 6H, OC(O)(CH₂)₃(CH₂CH═CH)₃(CH₂)₄CH₃), −0.60 (m, 4H, OC(O)CH₂CH₂CH₂). −0.65 (t, 8H, OC(O)CH₂), −0.90 (m, 4H, OC(O)CH₂CH₂). HRMS-ESI (m/z): [M+Na]⁺ calcd for M as C₆₈H₇₄N₈O₄Si, 1117.5500. found, 1117.5549.

Compound 7 is a blue solid. It is soluble in CH₂Cl₂, dimethylformamide, pyridine and toluene, slightly soluble in hexanes, and insoluble in methanol.

Compound 8: SiPc[OC(O)(CH═C(CH₃)CH═CH)₂C₆H₆(CH₃)₃]₂, Pc 301.

Under Ar, a mixture of SiPc(OH)₂ (20 mg, 0.030 mmol) and retinoic acid (50 mg, 0.17 mmol) was stirred at 190° C. for 3 h, diluted with CH₂Cl₂ (5 mL), filtered, and evaporated to dryness by rotary evaporation (room temperature). The solid was chromatographed (neutral Al₂O₃ III, CH₂Cl₂), washed (CH₃CN), vacuum dried (room temperature) and weighed (10 mg, 0.0090 mmol, 30%). UV-vis (toluene) λₘₐₓ, nm (log ε): 684 (5.4). NMR (CDCl₃): δ 9.67 (m, 8H, 1,4-Pc H), 8.37 (m, 8H, 2,3-Pc H), 5.82-6.00 (m, 4H, OC(O)CH═C(CH₃)CH═CHCH═C(CH₃)CH═CH), 5.75 (d, 2H, OC(O)CH═C(CH₃)CH═CHCH), 5.40 (d, 2H, OC(O)CH═C(CH₃)CH═CH), 4.65 (m, 2H, OC(O)CH═C(CH₃)CH), 2.80 (m, 2H, OC(O)CH), 1.85 (t, 4H, OC(O)(CH═C(CH₃)CH═CH)₂C₆H₆-3-CH₂), 1.55-1.40 (m, 16H, OC(O)(CH═C(CH₃)CH═CH)₂C₆H₆-2-CH₃, OC(O)(CH═C(CH₃)CH═CH)₂C₆H₆-4-CH₂ and OC(O)CH═C(CH₃)CH═CHCH═C(CH₃)), 1.35 (m, 4H, OC(O)(CH═C(CH₃)CH═CH)₂C₆H₆-5-CH₂), 0.82 (s, 12H, OC(O)(CH═C(CH₃)CH═CH)₂C₆H₆-6-(CH₃)₂), −0.20 (s, 6H, OC(O)CH═C(CH₃)).

Compound 8 is a blue solid It is soluble in CH₂Cl₂, dimethylformamide, pyridine and toluene, and insoluble in hexanes.

### Alkoxysilicon Phthalocyanines

Compound 9: SiPc[O(CH₂)₄CH₃]₂, Pc 307.

Under Ar, a mixture of SiPc(OH)₂ (20 mg, 0.030 mmol) and 1-pentanol (10 mL, 92 mmol) was refluxed for 6 h, diluted with CH₂Cl₂ (5 mL), filtered, and evaporated to dryness by rotary evaporation (room temperature). The solid was chromatographed (neutral Al₂O₃ III, CH₂Cl₂), washed (CH₃CN), vacuum dried (room temperature) and weighed (10 mg, 0.013 mmol, 45%). UV-vis (toluene) λₘₐₓ, nm (log ε): 674 (5.4). NMR (CDCl₃): δ 9.60 (m, 8H, 1,4-Pc H), 8.30 (m, 8H, 2,3-Pc H), −0.20 (t, 6H, CH₃), −0.42 (m, 4H, O(CH₂)₃CH₂), −1.40 (m, 4H, O(CH₂)₂CH₂), −1.75 (m, 4H, OCH₂CH₂), −2.18 (t, 4H, OCH₂). HRMS-ESI (m/z): [M+Na]⁺ calcd for M as C₄₂H₃₈N₈O₂Si, 737.2785. found, 737.2798.

Compound 9 is a blue solid. It is soluble in CH₂Cl₂, dimethylformamide, pyridine and toluene, and insoluble in hexanes and methanol. The structures of compounds 9-11 are shown in FIG. 5.

Compound 10: SiPc[OCH₂CH═C(CH₃)(CH₂)₂CH═C(CH₃)₂]₂, Pc 302.

Under Ar, a mixture of SiPc(OH)₂ (20 mg, 0.030 mmol) and geraniol (0.10 mL, 0.58 mmol) was stirred at 160° C. for 6 h, diluted with CH₂Cl₂ (5 mL), filtered, and evaporated to dryness by rotary evaporation (room temperature). The solid was chromatographed (neutral Al₂O₃ III, CH₂Cl₂), washed (CH₃CN), vacuum dried (room temperature) and weighed (10 mg, 0.010 mmol, 33%). UV-vis (toluene) λₘₐₓ, nm (log ε): 674 (5.4). NMR (CDCl₃): δ 9.60 (m, 8H, 1,4-Pc H), 8.30 (m, 8H, 2,3-Pc H), 4.50 (t, 2H, OCH₂CH═C(CH₃)(CH₂)₂CH), 1.45 (t, 2H, OCH₂CH), 1.40 (s, 6H, OCH₂CH═C(CH₃)(CH₂)₂CH═C(CH₃)), 1.30 (s, 6H, OCH₂CH═C(CH₃)(CH₂)₂CH═C(CH₃)CH₃), 1.15 (m, 4H, OCH₂CH═C(CH₃)CH₂CH₂), 0.70 (t, 4H, OCH₂CH═C(CH₃)CH₂), −0.02 (s, 6H, OCH₂CH═C(CH₃)), −1.20 (d, 4H, OCH₂). HRMS-ESI (m/z): [M+Na]⁺ calcd for M as C₅₂H₅₀N₈O₂Si, 869.3724. found, 869.3754.

Compound 10 is a blue solid. It is soluble in CH₂Cl₂, dimethylformamide, pyridine and toluene, and insoluble in hexanes and methanol.

Compound 11: SiPc[O(CH₂CH═C(CH₃)CH₂)₂CH₂CH═C(CH₃)₂]₂, Pc 303.

NaO(CH₂CH═C(CH₃)CH₂)₂CH₂CH═C(CH₃)₂. A mixture of farnesol (0.20 mL, 0.80 mmol) and Na (20 mg, 0.80 mmol) was stirred at room temperature for 1 h.

SiPc[O(CH₂CH═C(CH₃)CH₂)₂CH₂CH═C(CH₃)₂]₂, Pc 303, 10. Under Ar, a mixture of SiPc(OH)₂ (20 mg, 0.030 mmol) and NaO(CH₂CH═C(CH₃)CH₂)₂CH₂CH═C(CH₃)₂ (0.10 mL, 0.40 mmol) was stirred at 160° C. for 4 h, diluted with CH₂Cl₂ (5 mL), filtered, and evaporated to dryness by rotary evaporation (room temperature). The solid was chromatographed (neutral Al₂O₃ III, CH₂Cl₂), washed (CH₃CN), vacuum dried (room temperature) and weighed (12 mg, 0.0036 mmol, 12%). UV-vis (toluene) λₘₐₓ, nm (log ε): 674 (5.4). NMR (CDCl₃): δ 9.60 (m, 8H, 1,4-Pc H), 8.30 (m, 8H, 2,3-Pc H), 4.90 (t, 2H, 0(CH₂CH═C(CH₃)CH₂)₂CH₂CH), 4.50 (t, 2H, OCH₂CH═C(CH₃)CH₂CH₂CH), 1.80-1.60 (m, 8H OCH₂CH═C(CH₃)CH₂CH₂CH═C(CH₃)(CH₂)₂), 1.60-1.30 (m, 20H, OCH₂CH═C(CH₃)CH₂CH₂CH═C(CH₃)CH₂CH₂CH═C(CH₃)CH₂), 1.15 (m, 4H, OCH₂CH═C(CH₃)CH₂CH₂), 0.70 (t, 4H, OCH₂CH═C(CH₃)CH₂), −0.02 (s, 6H, OCH₂CH═C(CH₃)), −1.20 (d, 4H, OCH₂).

Compound 11 is a blue solid. It is soluble in CH₂Cl₂, dimethylformamide, pyridine and toluene, and insoluble in hexanes and methanol.

### Siloxysilicon Phthalocyanines

Compound 12: SiPc[OSi(CH═CH₂)₃]₂, Pc 279.

Under Ar, a mixture of SiPc(OH)₂ (50 mg, 0.080 mmol), trivinylethoxysilane (80 mg, 0.52 mmol) and pyridine (25 mL) was refluxed for 5 h, filtered, and evaporated to dryness by rotary evaporation (room temperature). The solid was washed (CH₃CN), vacuum dried (room temperature) and weighed (38 mg, 0.044 mmol, 55%). C₄₈H₃₈N₄O₂Si₃ Exact Mass: 786.23 UV-vis (toluene) λₘₐₓ, nm (log ε): 671 (5.5). NMR (CDCl₃): δ 9.60 (m, 8H, 1,4-Pc H), 8.30 (m, 8H, 2,3-Pc H), 4.40 (d, 6H, OSiCH═CH₂), 3.40 (m, 6H, OSiCH), 3.15 (d, 6H, OSiCH═CH₂). HRMS-ESI (m/z): [MαH]⁺ calcd for M as C₄₄H₃₄N₈O₂Si₃, 791.2191. found, 791.2200.

Compound 12 is a blue solid. It is soluble in CH₂Cl₂, dimethylformamide, pyridine and toluene, and insoluble in hexanes and methanol. The structure of compound 12 is shown in FIG. 6.

### Pc 12 Salts

SiPc[OSi(CH₃)₂(CH₂)₃NH(CH₃)₂]₂²⁺[OC(O)(CH₂)₁₇CH₃]₂⁻, Pc 287, 21.

Under Ar, a solution of Pc 12, SiPc[OSi(CH₃)₂(CH₂)₃N(CH₃)₂]₂, (5.0 mg, 0.0058 mmol), stearic acid (3.1 mg, 0.011 mmol) and CH₂Cl₂ (5 mL) was stirred at room temperature for 30 min and evaporated to dryness by rotary evaporation (room temperature). The solid was chromatographed (Biobeads, Hercules, Calif.; S—X3, CH₂Cl₂), vacuum dried (room temperature), and weighed (5.7 mg, 0.0042 mmol, 73%). UV-vis (toluene) λₘₐₓ, nm (log ε): 669 (5.5). NMR (CDCl₃): δ 9.60 (m, 8H, 1,4-Pc H), 8.35 (m, 8H, 2,3-Pc H), 2.00 (m, 4H, OC(O)CH₂), 1.80 (s, 12H, OSi(CH₃)₂(CH₂)₃N(CH₃)₂), 1.40 (m, 4H, OC(O)CH₂CH₂) 1.20 (m, 60H, OC(O)CH₂CH₂(CH₂)₁₅), 1.12 (t, 4H, OSi(CH₃)₂CH₂CH₂CH₂), 0.80 (t, 6H, OC(O)(CH₂)₁₇CH₃), −1.10 (t, 4H, OSi(CH₃)₂CH₂CH₂), −2.30 (t, 4H, OSi(CH₃)₂CH₂), −2.90 (s, 12H, OSi(CH₃)₂).

Compound 21 is a blue solid. It is soluble in ethanol, CH₂Cl₂, and dimethylformamide, moderately soluble in toluene, and insoluble in hexanes. The structures of compounds 21-27 are shown in FIG. 9.

Compound 22: SiPc[OSi(CH₃)₂(CH₂)₃NH(CH₃)₂]₂²⁺ [OC(O)(CH₂)₇CH═CH(CH₂)₇CH₃]₂⁻, Pc 288.

Under Ar, a solution of Pc 12, (5.0 mg, 0.0058 mmol), oleic acid (3.2 mg, 0.011 mmol) and CH₂Cl₂ (5 mL) was stirred at room temperature for 30 min and evaporated to dryness by rotary evaporation (room temperature). The solid was chromatographed (Biobeads, S—X3, CH₂Cl₂), vacuum dried (room temperature), and weighed (6.5 mg, 0.0048 mmol, 82%). UV-vis (toluene) λₘₐₓ, nm (log ε): 670 (5.5). NMR (CDCl₃): δ 9.60 (m, 8H, 1,4-Pc H), 8.35 (m, 8H, 2,3-Pc H), 5.30 (m, 4H, OC(O)(CH₂)₇CH═CH), 2.05 (m, 4H, OC(O)CH₂), 1.95 (m, 8H, OC(O)(CH₂)₆CH₂CH═CHCH₂), 1.80 (s, 12H, OSi(CH₃)₂(CH₂)₃N(CH₃)₂), 1.40 (m, 4H, OC(O)CH₂CH₂) 1.30-1.10 (m, 44H, OC(O)CH₂CH₂(CH₂)₄CH₂CH═CHCH₂(CH₂)₆CH₃ and OSi(CH₃)₂CH₂CH₂CH₂), 0.83 (t, 6H, OC(O)(CH₂)₇CH═CH(CH₂)₇CH₃), −1.10 (t, 4H, OSi(CH₃)₂CH₂CH₂), −2.30 (t, 4H, OSi(CH₃)₂CH₂), −2.90 (s, 12H, OSi(CH₃)₂).

Compound 22 is a blue solid. It is soluble in ethanol, CH₂Cl₂ and dimethylformamide, moderately soluble in toluene, and insoluble in hexanes.

Compound 23: SiPc[OSi(CH₃)₂(CH₂)₃NH(CH₃)₂]₂²⁺ [OC(O)(CH₂)₆(CH₂CH═CH)₂(CH₂)₄CH₃]₂, Pc 289.

Under Ar, a solution of Pc 12, (5.0 mg, 0.0058 mmol), linoleic acid (3.2 mg, 0.011 mmol) and CH₂Cl₂ (5 mL) was stirred at room temperature for 30 min and evaporated to dryness by rotary evaporation (room temperature). The solid was chromatographed (Biobeads, S—X3, CH₂Cl₂), vacuum dried (room temperature), and weighed (6.0 mg, 0.0042 mmol, 73%). UV-vis (toluene) λₘₐₓ, nm (log ε): 670 (5.5). NMR (CDCl₃): δ 9.60 (m, 8H, 1,4-Pc H), 8.35 (m, 8H, 2,3-Pc H), 5.30 (m, 8H, OC(O)(CH₂)₆(CH₂CH═CH)₂), 2.70 (t, 4H, OC(O)(CH₂)₇CH═CHCH₂), 2.00 (m, 12H, OC(O)CH₂(CH₂)₅CH₂CH═CHCH₂CH═CHCH₂), 1.79 (s, 12H, OSi(CH₃)₂(CH₂)₃N(CH₃)₂), 1.40-1.10 (m, 32H, OC(O)CH₂(CH₂)₅(CH₂CH═CH)₂CH₂(CH₂)₃) 1.15 (t, 4H, OSi(CH₃)₂CH₂CH₂CH₂), 0.83 (t, 6H, OC(O)(CH₂)₆(CH₂CH═CH)₂(CH₂)₄CH₃), −1.10 (t, 4H, OSi(CH₃)₂CH₂CH₂), −2.30 (t, 4H, OSi(CH₃)₂CH₂), −2.90 (s, 12H, OSi(CH₃)₂).

Compound 23 is a blue solid. It is soluble in ethanol, CH₂Cl₂ and dimethylformamide, moderately soluble in toluene, and insoluble in hexanes.

Compound 24: SiPc[OSi(CH₃)₂(CH₂)₃NH(CH₃)₂]₂²⁺ [OC(O)(CH₂)₆(CH₂CH═CH)₃CH₂CH₃]₂, Pc 290.

Under Ar, a solution of Pc 12, (7.3 mg, 0.0085 mmol), linolenic acid (4.7 mg, 0.017 mmol) and CH₂Cl₂ (5 mL) was stirred at room temperature for 30 min and evaporated to dryness by rotary evaporation (room temperature). The solid was chromatographed (Biobeads, S—X3, CH₂Cl₂), vacuum dried (room temperature), and weighed (9.0 mg, 0.0064 mmol, 75%). UV-vis (toluene) λₘₐₓ, nm (log ε): 669 (5.5). NMR (CDCl₃): δ 9.62 (m, 8H, 1,4-Pc H), 8.33 (m, 8H, 2,3-Pc H), 5.30 (m, 12H, OC(O)(CH₂)₆(CH₂CH═CH)₃), 2.75 (m, 8H, OC(O)(CH₂)₇(CH═CHCH₂)₂), 2.05-1.95 (m, 12H, OC(O)CH₂(CH₂)₅CH₂CH═CH(CH₂CH═CH)₂CH₂), 1.78 (s, 12H, OSi(CH₃)₂(CH₂)₃N(CH₃)₂), 1.30-1.10 (m, 20H, OC(O)CH₂(CH₂)₅) 1.15 (t, 4H, OSi(CH₃)₂CH₂CH₂CH₂), 0.95 (t, 6H, OC(O)(CH₂)₆(CH₂CH═CH)₃CH₂CH₃), −1.10 (t, 4H, OSi(CH₃)₂CH₂CH₂), −2.30 (t, 4H, OSi(CH₃)₂CH₂), −2.90 (s, 12H, OSi(CH₃)₂).

Compound 24 is a blue solid. It is soluble in ethanol, CH₂Cl₂ and dimethylformamide, moderately soluble in toluene, and insoluble in hexanes.

Compound 25: SiPc[OSi(CH₃)₂(CH₂)₃NH(CH₃)₂]₂²⁺ [OC(O)(CH₂)₂(CH₂CH═CH)₄(CH₂)₄CH₃]₂, Pc 291.

Under Ar, a solution of Pc 12, (5.0 mg, 0.0058 mmol), arachidonic acid (3.3 mg, 0.011 mmol) and CH₂Cl₂ (5 mL) was stirred at room temperature for 30 min and evaporated to dryness by rotary evaporation (room temperature). The solid was chromatographed (Biobeads, S—X3, CH₂Cl₂), vacuum dried (room temperature), and weighed (6.7 mg, 0.0046 mmol, 80%). UV-vis (toluene) λₘₐₓ, nm (log ε): 668 (5.5). NMR (CDCl₃): δ 9.62 (m, 8H, 1,4-Pc H), 8.33 (m, 8H, 2,3-Pc H), 5.30 (m, 16H, OC(O)(CH₂)₂(CH₂CH═CH)₄), 2.80-2.70 (m, 12H, OC(O)(CH₂)₃(CH═CHCH₂)₃), 2.10 (t, 4H, CH₂COO), 2.00 (m, 8H, OC(O)(CH₂)₂CH₂CH═CH(CH₂CH═CH)₂CH₂), 1.80 (s, 12H, OSi(CH₃)₂(CH₂)₃N(CH₃)₂), 1.50 (m, 4H, OC(O)CH₂CH₂), 1.40-1.20 (m, 12H, OC(O)(CH₂)₂(CH₂CH═CH)₄CH₂(CH₂)₃) 1.12 (t, 4H, OSi(CH₃)₂CH₂CH₂CH₂), 0.90 (t, 6H, OC(O)(CH₂)₂(CH₂CH═CH)₄(CH₂)₄CH₃), −1.10 (t, 4H, OSi(CH₃)₂CH₂CH₂), −2.30 (t, 4H, OSi(CH₃)₂CH₂), −2.90 (s, 12H, OSi(CH₃)₂).

Compound 25 is a blue solid. It is soluble in ethanol, CH₂Cl₂ and dimethylformamide, moderately soluble in toluene, and insoluble in hexanes.

Compound 26: SiPc[OSi(CH₃)₂(CH₂)₃NH(CH₃)₂]₂²⁺ [OC(O)(CH═C(CH₃)CH═CH)₂C₆H₆(CH₃)₃]₂, Pc 293.

Under Ar, a solution of Pc 12, (5.0 mg, 0.0058 mmol), retinoic acid (3.3 mg, 0.011 mmol) and CH₂Cl₂ (5 mL) was stirred at room temperature for 30 min and evaporated to dryness by rotary evaporation (room temperature). The solid was chromatographed (Biobeads, S—X3, CH₂Cl₂), vacuum dried (room temperature), and weighed (5.8 mg, 0.0041 mmol, 70%). UV-vis (toluene) λₘₐₓ, nm (log ε): 669 (5.5). NMR (CDCl₃): δ 9.61 (m, 8H, 1,4-Pc H), 8.30 (m, 8H, 2,3-Pc H), 6.80 (m, 2H, OC(O)CH), 6.20-6.00 (m, 10H, OC(O)CH═C(CH₃)CH═CHCH═C(CH₃)CH═CH), 2.12 (s, 6H, OC(O)CH═C(CH₃)), 2.00 (t, 4H, 3-CH₂), 1.92 (s, 6H, OC(O)CH═C(CH₃)CH═CHCH═C(CH₃)), 1.80 (s, 12H, OSi(CH₃)₂(CH₂)₃N(CH₃)₂), 1.65 (s, 6H, OC(O)(CH═C(CH₃)CH═CH)₂C₆H₆-2-CH₃), 1.60 (m, 4H, OC(O)(CH═C(CH₃)CH═CH)₂C₆H₆-4-CH₂), 1.42 (m, 4H, OC(O)(CH═C(CH₃)CH═CH)₂C₆H₆-5-CH₂), 1.10 (t, 4H, OSi(CH₃)₂CH₂CH₂CH₂), 1.00 (t, 12H, OC(O)(CH═C(CH₃)CH═CH)₂C₆H₆-6-(CH₃)₂), −1.10 (t, 4H, OSi(CH₃)₂CH₂CH₂), −2.28 (t, 4H, OSi(CH₃)₂CH₂), −2.90 (s, 12H, OSi(CH₃)₂).

Compound 26 is a blue solid. It is soluble in ethanol, CH₂Cl₂ and dimethylformamide, moderately soluble in toluene, and insoluble in hexanes.

Compound 27: SiPc[OSi(CH₃)₂(CH₂)₃NH(CH₃)₂]₂²⁺ [OC(O)CH₂(CH₂CH═CH)₆CH₂CH₃]₂⁻, Pc 292.

Under Ar, a solution of Pc 12, (3.3 mg, 0.0038 mmol), cis-4,7,10,13,16,19-docosahexaenoic acid (2.5 mg, 0.0076 mmol) and CH₂Cl₂ (5 mL) was stirred at room temperature for 30 min and evaporated to dryness by rotary evaporation (room temperature). The solid was chromatographed (Biobeads, S—X3, CH₂Cl₂), vacuum dried (room temperature), and weighed (3.7 mg, 0.0024 mmol, 64%). UV-vis (toluene) λₘₐₓ, nm (log ε): 670 (5.5). NMR (CDCl₃): δ 9.61 (m, 8H, 1,4-Pc H), 8.30 (m, 8H, 2,3-Pc H), 5.32 (m, 24H, OC(O)CH₂(CH₂CH═CH)₆), 2.80 (m, 20H, OC(O)(CH₂)₂(CH═CHCH₂)₅), 2.20 (m, 4H, OC(O)CH₂), 2.03 (m, 8H, OC(O)CH₂CH₂CH═CH(CH₂CH═CH)₅CH₂), 1.80 (s, 12H, OSi(CH₃)₂(CH₂)₃N(CH₃)₂), 1.15 (t, 4H, OSi(CH₃)₂CH₂CH₂CH₂), 0.95 (t, 6H, OC(O)CH₂(CH₂CH═CH)₆CH₂CH₃), −1.10 (t, 4H, OSi(CH₃)₂CH₂CH₂), −2.28 (t, 4H, OSi(CH₃)₂CH₂), −2.90 (s, 12H, OSi(CH₃)₂).

Compound 27 is a blue solid. It is soluble in ethanol, CH₂Cl₂ and dimethylformamide, moderately soluble in toluene, and insoluble in hexanes.

### 2,3-Disubstituted Phthalocyanines: MPc[2,3-O(C₂H₄CH(CH₃)CH₂)₂C₂H₄CH(CH₃)₂]₂

Compound 28: 3,7,11-Trimethyldodecanol.

Under a static H₂ atmosphere, a mixture of farnesol (7.5 g, 34 mmol), Raney nickel (2 g) and ethanol 50 mL stirred at room temperature for 1 week and filtered. The filtrate was evaporated to an oil by rotary evaporation (room temperature) and weighed (7.7 g, 34 mmol, 99%). NMR (CDCl₃): δ 3.69 (m, 2H, OCH₂), 1.70-1.00 (m, 17H, OCH₂(CH₂CH(CH₃)CH₂CH₂)₂CH₂CH), 0.85 (m, 12H, OCH₂(CH₂CH(CH₃)CH₂CH₂)₂CH₂CH(CH₃)₂).

Compound 28 is a colorless oil. It is soluble in CH₂Cl₂, dimethylformamide, toluene and hexanes.

Compound 29: 1-Bromo-3,7,11-trimethyldodecane.

A mixture of 28 (7.5 mg, 33 mmol), hydrobromic acid (48%, 100 mL, 884 mmol) and concentrated sulfuric acid (10.0 mL, 180 mmol) was refluxed for 4.5 h, diluted with H₂O, and extracted with ether (3 times, 20 mL each time). The extract was washed (10% aqueous NaHSO₃), dried (Na₂SO₄), and evaporated to an oil by rotary evaporation (room temperature). The oil was chromatographed (SiO₂, CHCl₃), vacuum dried (room temperature), and weighed (6.5 g, 22 mmol, 68%). NMR (CDCl₃): δ 3.45 (m, 2H, BrCH₂), 2.00-1.00 (m, 17H, OCH₂(CH₂CH(CH₃)CH₂CH₂)₂CH₂CH), 0.90 (m, 12H, OCH₂(CH₂CH(CH₃)CH₂CH₂)₂CH₂CH(CH₃)₂).

Compound 29 is a colorless oil. It is soluble in CH₂Cl₂, dimethylformamide, toluene and hexanes.

Compound 30: 1,2-Bis(3,7,11-trimethyldodecyloxy)benzene.

A mixture of 29 (6.2 g, 21 mmol) pyrocatecol (1.1 g, 10 mmol), K₂CO₃ (6.6 g, 48 mmol) and dimethylformamide (30 mL) was stirred at 70° C. for 5 h, diluted with H₂O (40 mL), and extracted with ether (3 times, 20 mL each time). The extract was washed (H₂O), dried (Na₂SO₄), and evaporated to an oil by rotary evaporation (room temperature). The oil was chromatographed (SiO₂, CH₂Cl₂), vacuum dried (room temperature), and weighed (3.2 g, 12 mmol, 60%). NMR (CDCl₃): δ 6.90 (s, 4H, 3,4,5,6-Ar H), 4.02 (m, 4H, OCH₂), 2.00-1.00 (m, 34H, OCH₂(CH₂CH(CH₃)CH₂CH₂)₂CH₂CH), 1.00-0.80 (m, 24H, OCH₂(CH₂CH(CH₃)CH₂CH₂)₂CH₂CH(CH₃)₂).

Compound 30 is a colorless oil. It is soluble in CH₂Cl₂, dimethylformamide, toluene and hexanes. It has the structure shown in formula:

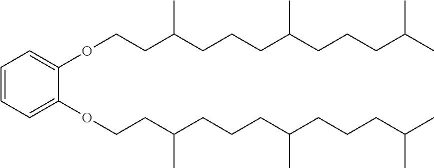

Compound 31: 1,2-Dibromo-4,5-bis(3,7,11-trimethyldodecyloxy)benzene.

A cooled (0° C.) solution of 30 (1.0 g, 1.8 mmol), I₂ (11 mg, 0.042 mmol) and CHCl₃ (1 mL) was treated with a solution of Br₂ (240 μL, 4.60 mmol) and CHCl₃ (1 mL) over 1 h, stirred at room temperature for 2 h, diluted with CHCl₃ (5 mL), washed (10% aqueous NaHSO₃), dried (Na₂SO₄), and evaporated to an oil by rotary evaporation (room temperature). The oil was chromatographed (SiO₂, CH₂Cl₂), vacuum dried (room temperature), and weighed (1.1 g, 1.6 mmol, 89%). NMR (CDCl₃): δ 7.05 (s, 2H, 3,6-Ar H), 3.97 (m, 4H, OCH₂), 1.90-1.00 (m, 34H, OCH₂(CH₂CH(CH₃)CH₂CH₂)₂CH₂CH), 1.00-0.80 (m, 24H, OCH₂(CH₂CH(CH₃)CH₂CH₂)₂CH₂CH(CH₃)₂).

Compound 31 is a pale-yellow oil. It is soluble in CH₂Cl₂, dimethylformamide, toluene and hexanes. It has the structure shown in formula:

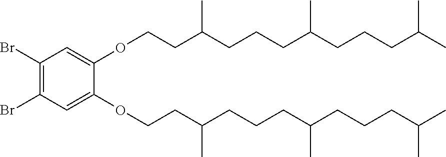

Compound 32: 4,5-Bis(3,7,11-trimethyldodecyloxy)phthalonitrile.

Under Ar, a mixture of 31 (790 mg, 1.20 mmol), CuCN (298 mg, 3.30 mmol) and dimethylformamide (20 mL) was refluxed for 5 h, treated with NH₄OH (10 mL), diluted with H₂O (10 mL), and extracted with CH₂Cl₂ (3 times, 15 mL each time). The extract was dried (Na₂SO₄), and evaporated to an oil by rotary evaporation (room temperature). The oil was chromatographed (SiO₂, CH₂Cl₂), and solid obtained was vacuum dried (room temperature), and weighed (667 mg, 0.540 mmol, 45%). NMR (CDCl₃): δ 7.12 (s, 2H, 3,6-Ar H), 4.09 (m, 4H, OCH₂), 2.00-1.00 (m, 34H, OCH₂(CH₂CH(CH₃)CH₂CH₂)₂CH₂CH), 1.00-0.80 (m, 24H, OCH₂(CH₂CH(CH₃)CH₂CH₂)₂CH₂CH(CH₃)₂).

Compound 32 is a white solid. It is soluble in CH₂Cl₂, dimethylformamide, toluene and hexanes. It has the structure shown in formula:

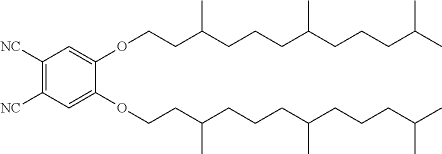

Compound 33: 4,5-Bis(3,7,11-trimethyldodecyloxy)-1,3-diiminoisoindoline.

Under Ar, a mixture of 32 (350 mg, 0.600 mmol), a solution of NaOCH₃ in CH₃OH (0.50 M, 0.4 mL, 0.22 mmol) and CH₃OH (10 mL) was stirred while being treated with a stream of NH₃ at room temperature for 1 h and then at reflux for 8 h. The reaction mixture was evaporated to dryness by rotary evaporation (room temperature), and the solid was washed (H₂O), vacuum dried (room temperature), and weighed (300 mg, 0.480 mmol, 84%). NMR (CDCl₃): δ 7.15 (s, 2H, 3,6-Ar H), 4.12 (m, 4H, OCH₂), 1.90-1.00 (m, 34H, OCH₂(CH₂CH(CH₃)CH₂CH₂)₂CH₂CH), 1.00-0.80 (m, 24H, OCH₂(CH₂CH(CH₃)CH₂CH₂)₂CH₂CH(CH₃)₂).

Compound 33 is a light-yellow solid. It is soluble in CH₂Cl₂, dimethylormamide, toluene and hexanes. It has the structure shown in formula:

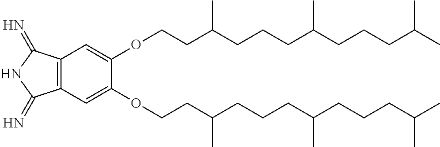

Compound 34: H₂Pc[2,3-O(C₂H₄CH(CH₃)CH₂)₂C₂H₄CH(CH₃)₂]₂, Pc 272. A mixture of 33 (300 mg, 0.48 mmol), chloroboron subphthalocyanine (68 mg, 0.16 mmol) and dimethylaminoethanol (10 mL) was stirred at 70° C. for 24 h, diluted with CH₃OH (35 mL) and filtered. The solid was extracted with CH₂Cl₂, evaporated to dryness by rotary evaporation (room temperature), washed (hot CH₃OH), air dried, and weighed (41 mg, 0.042 mmol, 27%). UV-vis (toluene) λₘₐₓ, nm (log ε): 694 (5.5), 658 (5.4). NMR (CDCl₃): δ 8.45 (m, 2H, Ar H), 8.12 (m, 2H, Ar H), 7.98 (m, 2H, Ar H), 7.75 (m, 2H, Ar H), 7.48 (m, 4H, Ar H), 7.28 (m, 2H, Ar H), 4.20 (m, 4H, OCH₂), 2.20 (m, 4H, OCH₂CH₂CH), 1.95 (m, 4H, OCH₂CH₂), 1.65-1.00 (m, 28H, OCH₂CH₂CH(CH₃)CH₂CH₂CH₂CH(CH₃)CH₂CH₂CH₂CH), 1.20 (d, 6H, OCH₂CH₂CH(CH₃)), 0.85 (d, 6H, OCH₂CH₂CH(CH₃)CH₂CH₂CH₂CH(CH₃)), 0.79 (d, 12H, OCH₂(CH₂CH(CH₃)CH₂CH₂)₂CH₂CH(CH₃)₂). HRMS-FAB (m/z): [M+H]⁺ calcd for M as C₆₂H₇₈N₈O₂, 967.6326. found, 967.6348.

Compound 34 is a dark-blue solid. It is soluble in CH₂Cl₂ and toluene, and slightly soluble in dimethylformamide and hexanes. It has the structure shown in formula:

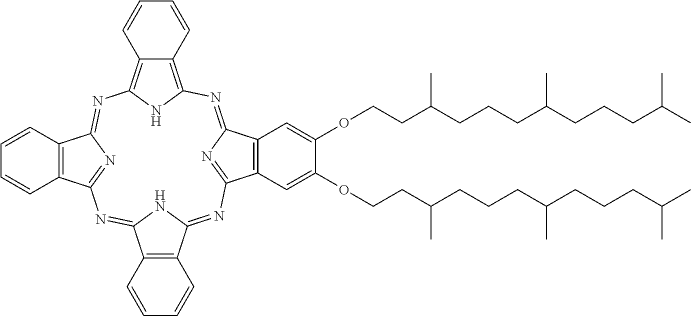

Compound 35: CoPc[2,3-O(C₂H₄CH(CH₃)CH₂)₂C₂H₄CH(CH₃)₂]₂, Pc 273.

A mixture of 34 (14 mg, 0.015 mmol), Co(CH₃CO₂)₂.4H₂O (29 mg, 0.12 mmol) and 1-hexanol (2 mL) was refluxed for 1 h, diluted with CH₃OH (3 mL) and filtered. The solid was chromatographed (SiO₂, CH₂Cl₂), vacuum dried (room temperature), and weighed (12 mg, 0.012 mmol, 80%). UV-vis (toluene) λₘₐₓ, nm (log ε): 664 (5.5). 35 is a dark-blue solid. It is slightly soluble in CH₂Cl₂, dimethylformamide, toluene, and hexanes. It has the structure shown in formula:

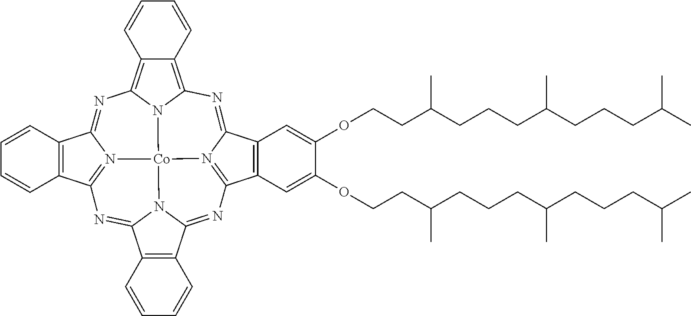

Compound 36: NiPc[2,3-O(C₂H₄CH(CH₃)CH₂)₂C₂H₄CH(CH₃)₂]₂, Pc 274. A mixture of 34 (16 mg, 0.016 mmol), Ni(CH₃CO₂)₂.4H₂O (30 mg, 0.12 mmol) and 1-hexanol (2 mL) was refluxed for 1 h, diluted with CH₃OH (3 mL) and filtered. The solid was chromatographed (SiO₂, CH₂Cl₂), vacuum dried (room temperature), and weighed (12 mg, 0.012 mmol, 73%). UV-vis (toluene) λₘₐₓ, nm (log ε): 665 (5.6). NMR (CDCl₃): δ 7.80 (m, 2H, Ar H), 7.62 (m, 2H, Ar H), 7.50 (m, 2H, Ar H), 7.30 (m, 2H, Ar H), 7.18 (m, 4H, Ar H), 6.60 (m, 2H, Ar H), 3.90 (m, 4H, OCH₂), 2.00 (m, 4H, OCH₂CH₂CH), 1.90-1.60 (m, 4H, OCH₂CH₂), 1.60-1.00 (m, 28H, OCH₂CH₂CH(CH₃)CH₂CH₂CH₂CH(CH₃)CH₂CH₂CH₂CH), 1.20 (d, 6H, OCH₂CH₂CH(CH₃)), 0.95 (d, 6H, OCH₂CH₂CH(CH₃)CH₂CH₂CH₂CH(CH₃)), 0.85 (d, 12H, OCH₂(CH₂CH(CH₃)CH₂CH₂)₂CH₂CH(CH₃)₂).

Compound 36 is a dark-blue solid. It is slightly soluble in CH₂Cl₂, dimethylformamide, toluene, and hexanes. It has the structure shown in formula:

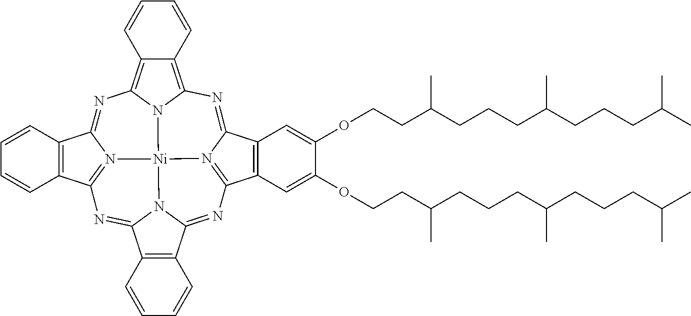

### Compound 37: ZnPc[2,3-O(C₂H₄CH(CH₃)CH₂)₂C₂H₄CH(CH₃)₂]₂, Pc 275.

A mixture of 34 (12 mg, 0.012 mmol), Zn(CH₃CO₂)₂.2H₂O (25 mg, 0.12 mmol) and 1-hexanol (2 mL) was refluxed for 1 h, diluted with CH₃OH (3 mL) and filtered. The solid was chromatographed (SiO₂, CH₂Cl₂), vacuum dried (room temperature), and weighed (10 mg, 0.010 mmol, 81%). UV-vis (toluene) λₘₐₓ, nm (log ε): 673 (5.6). 9.20 (m, 6H, 1,4-Ar H), 8.59 (m, 2H, 1,4-Ar H), 8.00 (m, 6H, 2,3-Ar H), 4.50 (m, 4H, OCH₂), 2.19 (m, 4H, OCH₂CH₂CH), 1.92 (m, 4H, OCH₂CH₂), 1.60-1.00 (m, 28H, OCH₂CH₂CH(CH₃)CH₂CH₂CH₂CH(CH₃)CH₂CH₂CH₂CH), 1.17 (d, 6H, OCH₂CH₂CH(CH₃)), 0.85 (d, 6H, OCH₂CH₂CH(CH₃)CH₂CH₂CH₂CH(CH₃)), 0.78 (d, 12H, OCH₂(CH₂CH(CH₃)CH₂CH₂)₂CH₂CH(CH₃)₂).

Compound 37 is a dark-blue solid. It is slightly soluble in CH₂Cl₂, dimethylformamide, toluene, and hexanes. It has the structure shown in formula:

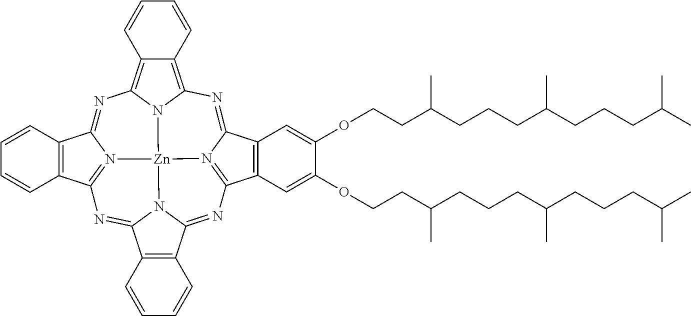

### Pc 12 and Pc 4 Analogues

SiNc[OSi(CH₃)₂(CH₂)₃N(CH₃)₂]₂, 41.

This compound was prepared according to the procedure described by Oleinick et al., Photochem. Photobiol., 57, (2), 242-247 (1993). Under Ar, a mixture of 40 (200 mg, 0.260 mmol) and CH₃OSi(CH₃)₂(CH₃)₂N(CH₃)₂ (300 mg, 1.80 mmol) and dry 2,6-lutidine (40 mL) was refluxed for 5 h, filtered, and evaporated to dryness by rotary evaporation (room temperature). The solid was washed (CH₃CN), vacuum dried (room temperature) and weighed (122 mg, 1.19 mmol, 45%). UV-vis (toluene) λₘₐₓ, nm (log ε): 776 (5.5). NMR (CDCl₃): δ 10.11 (s, 8H, 5,36-Nc H), 8.67 (m, 8H, 1,4-Nc H), 7.93 (m, 8H, 2,3-Nc H), 1.45 (s, 12H, OSi(CH₃)₂(CH₂)₃N(CH₃)₂), 0.95 (t, 4H, γ-CH₂), −0.81 (m, 4H, β-CH₂), −1.96 (m, 4H, α-CH₂), −2.52 (s, 12H, SiCH₃).

Compound 41 is a green solid. It is soluble in CH₂Cl₂, dimethylformamide, and toluene, and insoluble in hexanes. It has the structure shown in formula:

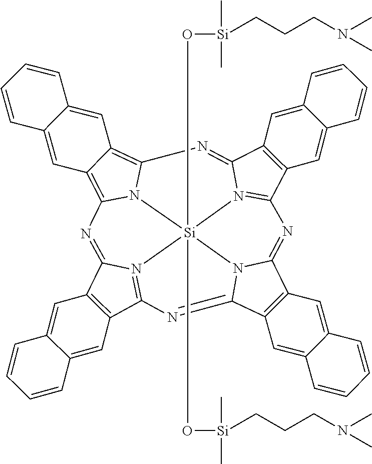

Compound 42: HOSiPcOSi(CH₃)₂(CH₂)₃N(C₂H₅)₂, Pc 34.

A mixture of SiPc[OSi(CH₃)₂(CH₂)₃N(C₂H₅)₂]₂ (Pc 41) (30 mg, 0.033 mmol), trichloroacetic acid (30 mg, 0.18 mmol) and CH₂Cl₂ (10 mL) was stirred at room temperature for 5 h. The resultant was mixed with pyridine (10 mL), diluted with H₂O (10 mL) and extracted with CH₂Cl₂ (3 times, 15 mL each time). The extract was dried (Na₂SO₄), and evaporated by rotary evaporation (room temperature). The solid was chromatographed (basic Al₂O₃ (V), CH₂Cl₂: ethyl acetate, 5:1), evaporated to dryness by rotary evaporation (room temperature), vacuum dried (room temperature), and weighed (20 mg, 0.027 mmol, 81%). UV-vis (toluene) λₘₐₓ, nm (log ε): 668 (5.4). NMR (CDCl₃): δ 9.21 (m, 8H, 1,4-Pc H), 8.20 (m, 8H, 2,3-Pc H), 1.70 (q, 2H, NCH₂), 0.85 (t, 2H, γ-CH₂), 0.50 (t, 3H, NCH₃), −1.30 (m, 2H, β-CH₂), −2.45 (m, 2H, α-CH₂), −3.05 (s, 3H, SiCH₃).

Compound 42 is a blue solid. It is soluble in CH₂Cl₂, dimethylformamide, and toluene, and insoluble in hexanes. It has the structure shown in formula:

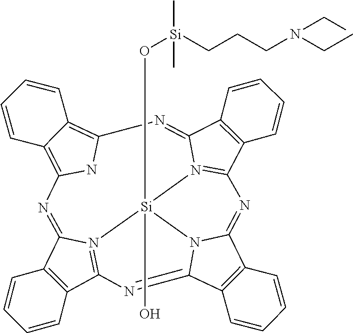

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A composition comprising a phthalocyanine photosensiziter or a salt thereof and an unattached free radical source.

2. The composition of claim 1, wherein the phthalocyanine compound has a structure of formula (I) or a salt thereof
[Pc-M] (I)
wherein Pc is a substituted or unsubstituted phthalocyanine; and
M is a diamagnetic metal ion, optionally complexed with or covalently bound to one or two axial ligands, wherein the metal ion is coordinated to the phthalocyanine moiety.

3. The composition of claim 1, wherein the phthalocyanine has a structure of formula (II) or 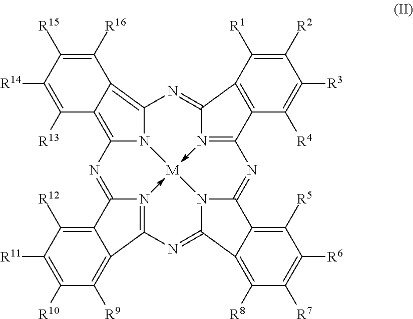
wherein M is a diamagnetic metal ion optionally complexed with or covalently bound to one or two axial ligands, wherein the metal ion is coordinated to the phthalocyanine moiety; and
R¹-R¹⁶ are each independently selected from hydrogen, halogen, nitro, cyano, hydroxy, thiol, amino, carboxy, aryl, heteroaryl, carbocyclyl, heterocyclyl, C₁₋₂₀alkyl, C₁₋₂₀alkenyl, C₁₋₂₀alkynyl, C₁₋₂₀alkoxy, C₁₋₂₀acyl, C₁₋₂₀alkylcarbonyloxy, C₁₋₂₀aralkyl, C₁₋₂₀hetaralkyl, C₁₋₂₀carbocyclylalkyl, C₁₋₂₀heterocyclylalkyl, C₁₋₂₀amino alkyl, C₁₋₂₀alkylamino, C₁₋₂₀thioalkyl, C₁₋₂₀alkylthio, C₁₋₂₀hydroxyalkyl, C₁₋₂₀alkyloxycarbonyl, C₁₋₂₀alkylaminocarbonyl, C₁₋₂₀alkylcarbonylamino, C₁₋₁₀alkyl-Z—C₁₋₁₀alkyl;
R¹⁷ is selected from hydrogen, C₁₋₂₀acyl, C₁₋₂₀alkyl, and C₁₋₂₀aralkyl; and
Z is selected from S, NR¹⁷, and O.

4. The composition of claim 3,
wherein M is (G)aY[(OSi(CH₃)₂(CH₂)_{b}N_{c}(R′)_{d}(R″)ₑ)_{f}Xg]p;
Y is selected from Si, Al, Ga, Ge, or Sn;
R′ is selected from H, CH₃, C₂H₅, C₄H₉, C₄H₈NH, C₄H₈N, C₄H₈NCH₃, C₄H₈S, C₄H₈O, C₄H₈Se, OC(O)CH₃, OC(O), CS, CO, CSe, OH, C₄H₈N(CH₂)₃CH₃, (CH₂)₂N(CH₃)₂, (CH₂)ₙN((CH₂)ₒ(CH₃))₂, and an alkyl group having from 1 to 12 carbon atoms;
R″ is selected from H, SO₂CH₃, (CH₂)₂N(CH₃)₂, (CH₂)₁₁CH₃, C(S)NHC₆H₁₁O₅, (CH₂)ₙN((CH₂)ₒ(CH₃))₂, and an alkyl group having from 1 to 12 carbon atoms;
G is selected from OH and CH₃;
X is selected from I, F, Cl, or Br;
a is 0 or 1;
b is an integer from 2 to 12;
c is 0 or 1;
d is an integer from 0 to 3;
e is an integer from 0 to 2;
f is 1 or 2;
g is 0 or 1;
n is an integer from 1 to 12;
o is an integer from 1 to 11; and
p is 1 or 2.

5. The composition of claim 4, wherein Y is Si or Al.

6. The composition of claim 5, wherein R¹, R⁴, R⁵, R⁸, R⁹, R¹², R¹³, and R¹⁶ are each independently selected from hydrogen, halogen, nitro, cyano, hydroxy, thiol, amino, and methyl; and
R², R³, R⁶, R⁷, R¹⁰, R¹¹, R¹⁴, and R¹⁵ are each independently selected from hydrogen, halogen, nitro, cyano, hydroxy, thiol, amino, carboxy, aryl, heteroaryl, carbocyclyl, heterocyclyl, C₁₋₆alkyl, C₁₋₆alkenyl, C₁₋₆alkynyl, C₁₋₆alkoxy, C₁₋₆acyl, C₁₋₆alkylcarbonyloxy, C₁₋₆carbocyclylalkyl, C₁₋₆-aminoalkyl, C₁₋₆alkylamino, C₁₋₆thioalkyl, C₁₋₆alkylthio, C₁₋₆hydroxyalkyl, C₁₋₆alkyloxycarbonyl, C₁₋₆alkylaminocarbonyl, and C₁₋₆alkylcarbonylamino.

7. The composition of claim 6, wherein M is selected from HOSiOSi(CH₃)₂(CH₂)₃N(CH₃)₂, Si[OSi(CH₃)₂(CH₂)₃N(CH₃)₂]₂, Si[OSi(CH₃)₂(CH₂)₃OCH₃]₂, and Si[OSi(CH₃)₂(CH₂)₃SCH₃]₂.

8. The composition of claim 3, wherein the free radical source is selected from 1,4-cyclohexadiene, 1,3-cyclooctadiene, 1,5-cyclooctadiene, linoleic acid, geraniol, farnesol, and squalene.

9. The composition of claim 1, wherein the composition comprises a pharmaceutical composition, the phthalocyanine is a pharmaceutically acceptable salt, and the composition further comprises a pharmaceutically acceptable carrier.

10. The pharmaceutical composition of claim 9, wherein Y is selected from the group consisting of bromide, chloride, sulfate, bisulfate, phosphate, nitrate, acetate, pyruvate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulphonate anions.

11. The pharmaceutical composition of claim 9, wherein the composition comprises a systemic formulation.

12. The pharmaceutical composition of claim 9, wherein the composition comprises a topical formulation.

13. A method of photodynamic therapy, comprising the steps of:
administering a therapeutically effective amount of a phthalocyanine photosensitizer to a subject, administering a discrete free radical source to the subject,
irradiating a target tissue in the subject with light having a wavelength suitable for excitation of the phthalocyanine photosensitizer.

14. The method of claim 13, wherein the target tissue is psoriatic tissue, eczemous tissue, or a tumor.

15. The method of claim 13, wherein the phthalocyanine photosensitizer has a structure of formula (II) or a salt thereof 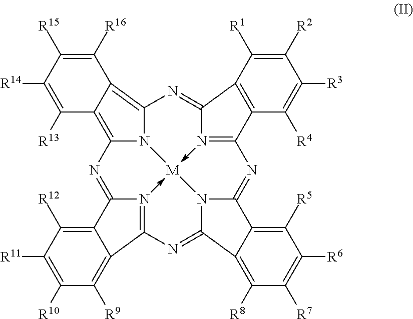
wherein M is a diamagnetic metal ion optionally complexed with or covalently bound to one or two axial ligands, wherein the metal ion is coordinated to the phthalocyanine moiety; and
R¹-R¹⁶ are each independently selected from hydrogen, halogen, nitro, cyano, hydroxy, thiol, amino, carboxy, aryl, heteroaryl, carbocyclyl, heterocyclyl, C₁₋₂₀alkenyl, C₁₋₂₀alkynyl, C₁₋₂₀alkoxy, C₁₋₂₀acyl, C₁₋₂₀alkylcarbonyloxy, C₁₋₂₀hetaralkyl, C₁₋₂₀carbocyclylalkyl, C₁₋₂₀heterocyclyl, C₁₋₂₀-aminoalkyl, C₁₋₂₀alkylamino, C₁₋₂₀alkylthio, C₁₋₂₀hydroxyalkyl, C₁₋₂₀alkyloxycarbonyl, C₁₋₂₀alkylaminocarbonyl, C₁₋₂₀alkylcarbonylamino, C₁₋₁₀alkyl-Z—C₁₋₁₀alkyl;
R¹⁷ is selected from hydrogen, C₁₋₂₀acyl, C₁₋₂₀alkyl, and C₁₋₂₀aralkyl; and
Z is selected from S, NR¹⁷, and O.

16. The method of claim 15,
wherein M is (G)aY[(OSi(CH₃)₂(CH₂)_{b}N_{c}(R′)_{d}(R″)ₑ)_{f}Xg]p;
Y is selected from Si, Al, Ga, Ge, or Sn;
R′ is selected from H, CH₃, C₂H₅, C₄H₉, C₄H₈NH, C₄H₈N, C₄H₈NCH₃, C₄H₈S, C₄H₈O, C₄H₈Se, OC(O)CH₃, OC(O), CS, CO, CSe, OH, C₄H₈N(CH₂)₃CH₃, (CH₂)₂N(CH₃)₂, (CH₂)ₙN((CH₂)ₒ(CH₃))₂, and an alkyl group having from 1 to 12 carbon atoms;
R″ is selected from H, SO₂CH₃, (CH₂)₂N(CH₃)₂, (CH₂)₁₁CH₃, C(S)NHC₆H₁₁O₅, (CH₂)ₙN((CH₂)ₒ(CH₃))₂, and an alkyl group having from 1 to 12 carbon atoms;
G is selected from OH and CH₃;
X is selected from I, F, Cl, or Br;
a is 0 or 1;
b is an integer from 2 to 12;
c is 0 or 1;
d is an integer from 0 to 3;
e is an integer from 0 to 2;
f is 1 or 2;
g is 0 or 1;
n is an integer from 1 to 12;
o is an integer from 1 to 11; and
p is 1 or 2.

17. The method of claim 13, wherein the free radical source is a polyunsaturated alkene or cycloalkene.

18. The method of claim 13, wherein the free radical source is selected from 1,4-cyclohexadiene, 1,3-cyclooctadiene, 1,5-cyclooctadiene, linoleic acid, geraniol, farnesol, and squalene.

19. A phthalocyanine photosensitizer wherein the phthalocyanine compound has a structure of formula (I) or a salt thereof
[Pc-M] (I)
wherein Pc is a substituted or unsubstituted phthalocyanine; and
M is a diamagnetic metal ion, optionally complexed with or covalently bound to one or two axial ligands, wherein the metal ion is coordinated to the phthalocyanine moiety,
and wherein the phthalocyanine compound further comprises a free radical source.

20. The phthalocyanine photosensitizer of claim 19, wherein the phthalocyanine photosensitizer has a structure according to formula (II): 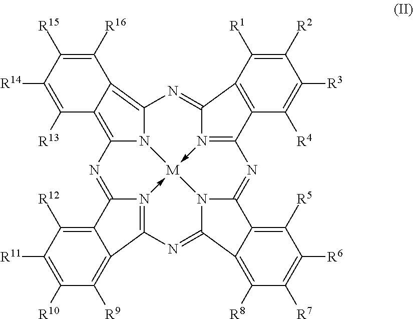
wherein M is a diamagnetic metal ion optionally complexed with or covalently bound to one or two axial ligands, wherein the metal ion is coordinated to the phthalocyanine moiety; and
R¹-R¹⁶ are each independently selected from hydrogen, halogen, nitro, cyano, hydroxy, thiol, amino, carboxy, aryl, heteroaryl, carbocyclyl, heterocyclyl, C₁₋₂₀alkyl, C₁₋₂₀alkenyl, C₁₋₂₀alkynyl, C₁₋₂₀alkoxy, C₁₋₂₀acyl, C₁₋₂₀alkylcarbonyloxy, C₁₋₂₀hetaralkyl, C₁₋₂₀carbocyclylalkyl, C₁₋₂₀heterocyclylalkyl, C₁₋₂₀-aminoalkyl, C₁₋₂₀alkylamino, C₁₋₂₀alkylthio, C₁₋₂₀hydroxyalkyl, C₁₋₂₀alkyloxycarbonyl, C₁₋₂₀alkylaminocarbonyl, C₁₋₂₀alkylcarbonylamino, C₁₋₁₀alkyl-Z—C₁₋₁₀alkyl;
R¹⁷ is selected from hydrogen, C₁₋₂₀acyl, C₁₋₂₀alkyl, and C₁₋₂₀aralkyl; and
Z is selected from S, NR¹⁷, and O, and wherein the free radical source is selected from unsaturated carboxy ligands, unsaturated alkoxy ligands, unsaturated siloxy ligands, and
thiomethoxysiloxy ligands and is substituted on one or more of the axial ligands or R¹-R¹⁶.

21. A method of photodynamic therapy, comprising the steps of:
administering a therapeutically effective amount of a phthalocyanine photosensitizer including a free radical source to the subject,
irradiating a target tissue in the subject with light having a wavelength suitable for excitation of the phthalocyanine photosensitizer.

22. The method of claim 21, wherein the target tissue is psoriatic tissue, eczemous tissue, or a tumor.

23. The method of claim 21, wherein the phthalocyanine photosensitizer has a structure according to the formula of claim 20.
